(19) United States
(12) Patent Application Publication
(10) Pub. No.: US 2026/0218113 A1
(43) Pub. Date: Jul. 30, 2026

(54) PROCESSES AND SYSTEMS FOR CULTURING ALGAE

(71) Applicant: Neste Oyj, Espoo (FI)

(72) Inventors: Jeffrey KANEL, Porvoo (FI); David BRYANT, Porvoo (FI); Cecil CHURN III, Porvoo (FI)

(21) Appl. No.: 19/143,722

(22) PCT Filed: Dec. 22, 2023
(86) PCT No.: PCT/FI2023/050733

§ 371 Date: Jun. 26, 2025

Related U.S. Application Data
(60) Provisional application No. 63/477,841, filed on Dec. 30, 2022.

(30) Foreign Application Priority Data
Mar. 21, 2023 (FI) 20235330

(51) Int. Cl. C12N 1/12; C12M 1/00; C12M 1/34

(52) U.S. Cl. CPC C12N 1/12; C12M 21/02; C12M 23/18; C12M 41/34

(57) ABSTRACT

Processes are disclosed for culturing algae by maintaining aerobic conditions and controlling algal age in an algal growth system. The processes can include exposing an aquaculture medium including microorganisms capable of phototaxis to one or more light sources in an illumination zone of at least one algal aquaculture zone; allowing the microorganisms capable of phototaxis in the aquaculture medium to move toward the one or more light sources, thereby forming an upper layer containing the microorganisms capable of phototaxis and a lower layer in the illumination zone; separating at least part of the upper layer from at least the lower layer in a separation zone; and transferring at least part of the microorganisms capable of phototaxis of the upper layer from the separation zone to a harvesting zone. Systems for performing the processes described herein are also disclosed.

### FIELD

The present disclosure relates to processes and systems for culturing algae by controlling algae age and/or maintaining aerobic conditions in an algal growth system.

### BACKGROUND INFORMATION

Algae as a source of foodstuffs and energy has been recognized for decades, however realization has remained elusive. One of the reasons for the lack of algae-based sources of food and energy is due to challenges associated with algal culture growth. Algae have a reputation for being difficult to grow in open ponds or tanks since they quickly run out of carbon dioxide, which is needed for their growth, in these environments or fall prey to microscopic predators. Current processes to handle this challenge involve circulating the algal culture rapidly through wide, flat tubes of thin plastic.

Many types of algal bioreactors have been proposed to address the issues encountered with algal culture growth. These algal bioreactors generally fall into three categories: fermentation units, enclosed photobioreactors, and open-pond bioreactors.

Fermentation units are considered for the growth of genetically modified algae that are heterotrophic. The fermentation unit can be constructed of steel and involves sophisticated process control. This type of algal bioreactor can be employed for high-value products, for example, docosahexaenoic acid (DHA), produced by Martek Biosciences of Columbia, Maryland, currently a subsidiary of DSM. However, this type of bioreactor is extremely expensive for the production of lower-value chemicals, for example, biofuels because of the enormous capital cost of the fermentation equipment. Furthermore, the fermentation process employed by these bioreactors can involve a source of sugar, which adds additional costs, for example, when lower-value products are being produced.

Enclosed photobioreactors have also been proposed for the production of biofuels. These photobioreactors can be transparent so that the algae they contain can utilize sunlight as a source of energy. These enclosed photobioreactors may include plastic bags, glass and plastic tubes, ponds in green-house structures, and the like. Tubular reactors were popularized by Greenfuel Technologies Corporation of Cambridge, Massachusetts for the production of biofuels, but the technology was economically unsuccessful. Plastic bag bioreactors are typified by those utilized by Algenol Biofuels of Bonita Springs, Florida. Although the capital cost of constructing a bioreactor from plastic instead of steel is substantially reduced, this type of bioreactor is still so expensive that the only major commercial use is for the production of, for example, astaxanthin, a high-value carotenoid. Furthermore, the algae can foul the plastic or glass surface, thus attenuating the essential light. Thus, the use of enclosed photobioreactors is only of commercial interest for the production of high-value products.

Open-pond bioreactors have been classified as natural, intensive, and extensive. The natural open-pond bioreactors are defined as those naturally occurring ponds where the conditions are right to grow algae. These ponds may contain either fresh or saline water, and they are unmanaged in terms that they lack fertilizer addition and mechanical agitation. Natural open ponds that contain algae are common along the shores of the Great Salt Lake in Utah. The lack of control of the algal growth in these ponds can make them unsuitable for commercial use.

Both the intensive and extensive modes of aquaculture involve the addition of fertilizers to the medium to supply necessary inorganic nutrients, phosphorus, nitrogen, iron, and trace metals that are necessary for biomass production through photosynthesis. The primary difference between the two modes of production is mixing of the growth medium. Intensive ponds employ mechanical mixing devices while extensive ponds rely on adventitious mixing by the wind. Therefore, factors that affect algae growth can be more accurately controlled in intensive aquaculture.

Intensive aquaculture ponds have been constructed of concrete blocks and are lined with plastic. Brine depth generally is controlled at 20 centimeters, which has been considered to be an optimum depth for producing algal biomass. A number of configurations of these ponds have also been proposed. However, the open-air raceway ponds are important commercially. Raceway ponds employ paddle wheels to provide mixing. Chemical and biological parameters are carefully controlled, including salt and fertilizer concentrations, pH of the brine, and purity of the culture.

Extensive aquaculture has been practiced in the hot and arid regions of Australia for the production of beta-carotene. Outdoor ponds for extensive aquaculture generally are larger than those for intensive aquaculture and normally are constructed in lake beds. Open-air ponds have been bounded by earthen dikes. No mixing devices are employed.

Nielson (U.S. Pat. No. 4,958,460) discloses a process and system for growing and harvesting algae of the genus Dunaliella, along with their associated bacteria. There is no recognition of any opportunity to selectively concentrate algae during periods when they concentrate at the surface, nor are measuring and monitoring the aerobic-anaerobic condition of the growth medium to avoid algae colony collapse disclosed.

Other reasons for the lack of progress in growing algae commercially is due in part to fluctuating crude oil prices and the equally, challenging issue of finding solutions to the multitude of challenges associated with commercial scale operation of an algal growth facility.

An issue encountered with commercial scale operations for algal growth is maintaining the algae colony viable and avoiding a situation referred to a colony collapse. Colony collapse can occur when the algae need oxygen for aquatic respiration and sufficient oxygen is unavailable. The presence of oxygen in an aqueous medium has been referred to as an aerobic medium and an aqueous medium with a deficiency or lack of oxygen is referred to as an anaerobic medium. Thus, to keep an algae colony viable, conditions must be maintained aerobic.

Another complication with scaling up algal growth cultures for commercial use is that bacteria coexist with the algae and utilize oxygen that algae respire when exposed to radiation, for example, that from the sun or other suitable artificial illumination sources. Being plants, algae intake carbon dioxide and expire oxygen. During daylight hours, algae migrate, using flagella, towards the light whereas the bacteria remain uniformly distributed throughout the aqueous medium due to their smaller size and Brownian Motion that causes the bacteria to move in random directions. Migration towards light is termed phototaxis. During daylight competition for oxygen is not a serious concern. However, at night algae no longer have the sun as a source of energy, and their carbon dioxide respiration decreases or ceases. To survive they consume stored energy in the form of carbohydrates and/or oils that lie within their cells. For this process, they require oxygen. Thus, both bacteria and algae compete for oxygen when illumination is absent.

In summary, while illuminated algae do not compete with bacteria for oxygen, in the dark they need oxygen to survive. Therefore, if the algal culture contains a high number of bacteria, the bacteria can out-compete the algae for oxygen during dark hours, thus ultimately leading to colony collapse. That is, the bacteria can create an anaerobic environment for the algae. Under anaerobic conditions, the algae colony will collapse.

The development of culturing processes and equipment that can overcome at least some of the challenges and complications would be a benefit to the foodstuff and energy industries. There is an increasing interest in utilizing algal biomass for a plethora of sustainable activities, for example, a source of renewable energy, a mode to capture carbon dioxide safely and efficiently from the atmosphere for carbon sequestration, and a renewable source of chemical intermediates.

### SUMMARY

A process disclosed herein to culture algae by controlling algae age and/or maintaining aerobic conditions in an algal growth system includes at least one or more of the following: feeding an aquaculture medium into at least one algal aquaculture zone, exposing the aquaculture medium including microorganisms capable of phototaxis to one or more light sources in an illumination zone of the at least one algal aquaculture zone, wherein the microorganisms include algae; allowing the microorganisms capable of phototaxis to move toward the one or more light sources in the aquaculture medium, thereby forming an upper layer containing the microorganisms capable of phototaxis and a lower layer in the illumination zone; separating at least part of the upper layer from at least part of the lower layer in a separation zone; and transferring at least part of the microorganisms capable of phototaxis of the upper layer from the separation zone to a harvesting zone.

A system disclosed herein to culture algae by controlling algae age and/or maintaining aerobic conditions includes at least one or more of the following components: (i) one or more algal aquaculture zones, the one or more algal aquaculture zones including an aquaculture medium containing microorganisms capable of phototaxis; (ii) an illumination zone in communication with the one or more algal aquaculture zones; (iii) a separation zone in communication with the one or more algal aquaculture zones; (iv) a harvesting zone in communication with the separation zone; and/or (v) optionally a lighting system in communication with at least the one or more algal aquaculture zones, the illumination zone(s) and/or the separation zone.

A system disclosed herein to culture algae by controlling algae age and/or maintaining aerobic conditions includes at least one or more of the following: (i) an aquaculture pretreatment unit configured for conditioning an aqueous medium with at least algal nutrients to obtain the aquaculture medium, wherein the aquaculture pretreatment unit is arranged to feed the aquaculture medium containing microorganisms capable of phototaxis to one or more aquaculture zones optionally by gravity feed; (ii) a pipe, a tube and/or a canal, optionally a covered canal, in communication with a harvesting zone, wherein the pipe, tube and/or canal is optionally arranged to feed the microorganisms capable of phototaxis to the harvesting zone; (iii) a biological polishing system containing a population of microorganisms that metabolize glycerol, optionally in communication with the harvesting zone and/or the one or more aquaculture zones optionally by at least one or more of a pipe, a tube and/or a canal; (iv) one or more ponds for crystallizing salt, optionally in communication with the biological polishing system, wherein the one or more aquaculture zones include at least one algal aquaculture zone containing: the aquaculture medium, an illumination zone in communication with the at least one algal aquaculture zone, a separation zone in communication with the at least one algal aquaculture zone and the pipe, tube and/or canal in communication with the harvesting zone; and optionally a lighting system in communication with the at least the one algal aquaculture zone, the illumination zone and/or the separation zone.

A process for culturing and harvesting algae disclosed herein comprises using the system of the present disclosure.

Also, disclosed herein is use of the system of the present disclosure for culturing and harvesting algae and/or for controlling algae age.

### BRIEF DESCRIPTION OF THE DRAWINGS

Some of the features and advantages of the disclosure have been stated. Other advantages will become apparent as the description of the disclosure proceeds, taken in conjunction with the accompanying drawings, in which:

FIG. 1 depicts an exemplary process for maintaining aerobic conditions and/or controlling algae age in an aquaculture zone.

FIG. 2 depicts an exemplary system for maintaining aerobic conditions and/or controlling algae age in an aquaculture zone. The system is designed to remove biomass material from the surface of the aquaculture zone and/or the illumination zone by skimming the biomass material over a weir into a separation zone.

FIG. 3 depicts an exemplary system for maintaining aerobic conditions and/or controlling algae age in an aquaculture zone. The system is designed to remove biomass material from the surface of the aquaculture zone and/or the illumination zone by pumping the biomass material to a filtering unit. The filtering unit separates the biomass material from the remainder of the aquaculture medium via a filter and transports the separated biomass material to a harvesting zone.

FIG. 4 depicts an exemplary system for maintaining aerobic conditions and/or controlling algae age in an aquaculture zone. The system is designed to remove biomass material from the surface of the aquaculture zone and/or the illumination zone by pumping the biomass material to a sedimentation unit. The sedimentation unit separates the biomass material from the remainder of the aquaculture medium via a sedimentation process and transports the separated biomass material to a harvesting zone.

FIG. 5 depicts a plot representing stable aerobic operation with EMF Eb monitoring.

FIG. 6 depicts a plot representing a circumstance where there has been a steady decline in the dissolved oxygen concentration with EMF Eb monitoring.

### DETAILED DESCRIPTION

### Methods/Processes

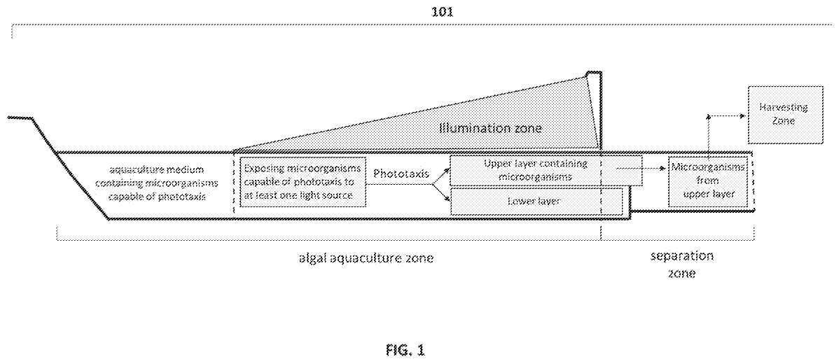

A process 101 for culturing algae by maintaining aerobic conditions and/or controlling algae age in an algal growth system is depicted in FIG. 1.

The process 101 includes feeding an aquaculture medium into at least one algal aquaculture zone.

### Aquaculture Medium

In exemplary embodiments, the aquaculture medium has an elevated salinity, for example, a salinity of about 5 wt % to about 27 wt %. For example, the aquaculture medium can have a salinity that is about 5% or greater than 5%, about 6% or greater than 6%, about 7% or greater than 7%, at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, at least about 25 wt %, at least about 26 wt %, or at least about 27 wt %. In exemplary embodiments the aquaculture medium is saturated with salt. In other exemplary embodiments, the aquaculture medium can have a salinity from about 7 wt % to saturation, from about 10 wt % to saturation, from about 15 wt % to saturation, from about 20 wt % to saturation, from about 5 wt % to about 25 wt %, from about 5 wt % to about 20 wt %, from about 10 wt % to about 20 wt %, from about 5 wt % to about 15 wt %, from about 10 wt % to about 15 wt %, or from about 5 wt % to about 10 wt %.

The expression “salinity” refers to the total amount of dissolved salts that can be present within the biomass material and/or the medium in which the biomass material resides, for example, an aqueous stream. Salts which can be dissolved and found in the biomass material and/or its medium include, but are not limited to, those found in natural waters, for example, sodium chloride, magnesium chloride, calcium and magnesium sulfates, bicarbonates, and carbonates. In more general terms, salinity is indicated by the water source, for example, a freshwater, a brackish water, a saline water, and a brine. Ranges of salinity are associated with these general terms and these ranges are defined as <0.05 wt % for freshwater, 0.05-3 wt % for brackish water, 3-5 wt % for saline water, and >5 wt % for a brine.

As used herein, wt % refers to a dry mass of a component in a solution in grams divided by 100 grams of the solution.

The aquaculture medium can include water and salts that are found in seawater, terminal lakes, or aquifers. The aquaculture medium may include combinations of ions found in seawater. The concentration of the salts in the aquaculture medium may range from just a trace to saturation so that suitable names to describe the salinity of the aquaculture medium would range from fresh water, brackish water, salt water, brine, and saturated brine, respectively, as the salts in the aquaculture medium increase. The desired concentration of the salts in the aquaculture medium will depend on the type of algae that are being grown in the algal aquaculture zone and cover the range from fresh water to saturated brine.

Various combinations of ions found in seawater may be included within the aquaculture medium, for example, ion combinations derived from one or more of the following sources including: water derived from streams, lakes, rivers, or other sources associated with fresh water; water derived from underground aquifers that may include various ion concentrations; water derived from industrial, agricultural, or municipal sources that may or may not have received treatment; or water derived from brackish sources where fresh water is combined with sea water or ocean water in various proportions; sea water or ocean water that may be derived from the various seas and oceans located around the globe; or combinations thereof. The combination of ions for the aquaculture medium may be derived directly from these sources or may be derived by evaporating the desired amount of water from any of these sources to leave the desired ion-rich solution for use in the aquaculture medium.

The ion combination that can be included with the aquaculture medium may differ for different algae, an example of which is disclosed in U.S. Pat. No. 6,986,323, the contents of which are incorporated herein by reference in their entirety. Combination of ions can be obtained by the evaporation of sea water or ocean water, including the evaporation of ancient sea waters that form terminal lakes, for example, the Great Salt Lake in Utah and that form various aquifers. The combination of ions can also be derived from crystallizers, for example, those wherein sodium chloride ions are precipitated.

The aquaculture medium can also be aerobic and/or anaerobic. An aquaculture medium with oxygen is termed aerobic; wherein one without oxygen is termed anaerobic. Oxygen levels or the aerobic/anaerobic status of the aquaculture medium can be determined by monitoring the dissolved oxygen concentration. Processes of measuring dissolved oxygen in the aquaculture medium can include the use of dissolved oxygen sensors or probes and their associated electronics.

“Dissolved oxygen sensor” as used herein refers to an entire sensor assembly including the electrodes, electrolyte solutions, membranes and thermistor thermometers, light source, photomultipliers, and signal amplifiers.

Dissolved oxygen sensors that measure a current as oxygen is reduced at a cathode are those that involve galvanic means (Mancy & Westgarth 1962) and those that involve polargraphic means (Carritt & Kanwisher 1959). Both can be used in the systems and processes disclosed herein and both use an electrode system where dissolved oxygen reacts at the cathode to produce a current. This current is commonly expressed as the oxidation reduction potential (ORP). ORP is measured in millivolts (mV). Like pH, it is not a measurement of concentration directly, but of activity level. In a solution with a single component (oxygen), ORP indicates concentration. As long as the oxidation-reduction potential (ORP) is maintained at positive Eh values, where Eh is a measure of the redox (oxidation-reduction) state of solutes in a solution, and the aqueous medium remains aerobic and therefore suitable for healthy algae growth.

A third general class of dissolved oxygen sensors that can be used in the systems and processes disclosed herein are those that use fluorescence quenching, for example, thus containing metal organic ruthenium complexes (Hasumoto 2006). These sensors generally include a sensor tip, optical fibers, an exciting light source, a photomultiplier, and a signal amplifier. The intensity of the fluorescence emitted is a function of the dissolved oxygen concentration. The biggest advantages of fluorescence-based sensors are inherent reliability and low maintenance. Low maintenance and the absence of membranes or electrolyte are key advantages of the fluorescent-based oxygen sensors.

The at least one dissolved oxygen sensor can be configured to measure the galvanic current and/or track millivoltage levels in the one or more algal aquaculture zones and/or illumination zone. The at least one dissolved oxygen sensor can also be configured to measure oxidation-reduction potentials in the one or more algal aquaculture zones.

The dissolved oxygen concentration of the aquaculture medium can be expressed as milligrams of oxygen per liter of water (mg/L), milliliters of oxygen per liter of water (ml/L), parts per million (ppm) or as percent saturation (% sat). Dissolved oxygen sensors can be used to measure the partial pressure of oxygen in the aquaculture medium. Oxygen pressure is dependent on both the salinity and temperature of the aquaculture medium. Increasing the temperature of the aquaculture medium reduces the solubility of oxygen. Increasing the salinity of the aquaculture medium likewise decreases the solubility of oxygen.

The aquaculture medium can also possess a biochemical oxygen demand comprising organic matter that does not include the contribution of living algae, archaea, or bacteria. Bacteria can feed on this organic matter in the aquaculture medium. Biochemical oxygen demand (BOD) is the amount of oxygen consumed by algae, archaea, bacteria and other organisms while they consume organic matter under aerobic conditions, and it is measured by EPA Analytical Method 405.1. If the level of organic matter (BOD) becomes excessive, bacteria consume a disproportionate quantity of the oxygen leaving little for algae that need oxygen when not exposed to light. In the absence of sufficient oxygen, algae colony collapse may result. In general, for every kilogram of BOD present in the pond, it takes 1.25 kilograms of O₂, and this adds to the oxygen demand at night. Since the dissolved oxygen content in the algal aquaculture zone can be measured in terms of mg oxygen/liter of media, then the available oxygen for treating the BOD can be known. It is important to keep the dissolved oxygen content above zero in the algal aquaculture zone at night to assure that the algae have sufficient oxygen available to prevent colony collapse.

In exemplary embodiments, the process for maintaining aerobic conditions and/or controlling algae age in the at least one algal aquaculture zone includes controlling the organic matter level (BOD) and/or the algae to bacteria level in the aquaculture medium to maintain the dissolved oxygen content of the aquaculture pond to be above zero at night in order to maintain the algae health.

In exemplary embodiments, algae from divisions Bacillariophyta, Chlorophycophyta, Chrysophycophyta, Cyanophycophyta, Cryptophycophyta, Phaeophycophyta,

Pyrrhophycophyta, Rhodophycophyta or combinations thereof are suitable for use in the present disclosure.

The algae or microalgae used can include, but are not limited to, one or more species from the following genera: Acutodesmus, Achnahtes, Amphipora, Amphora, Anabaena, Ankistrodesmus, Arthrospira (also known as Spirulina), Asteromonas, Asterionella, Boekelovia, Borodinella, Botryococcus, Bracteacoccus, Carteria, Chaetoceros, Chlamydomonas, Chlorella, Chlorococcum, Chlorogonium, Chloromonas, Chroomonas, Chrysophaera, Ceratium, Closterium, Coccolithus, Coelastrella, Coscinodiscus, Cosmarium, Cricosphaera, Crocosphaera, Crypthecodinium, Cryptomonas, Cyanocystis, Cyanospira, Cyclotella, Desmodesmus, Ditylum, Dunaliella, Ellipsoidon, Emiliania, Eremosphaera, Euglena, Fragilaria, Franceia, Galdieria, Gracilaria, Graesiella, Guinardia, Haematococcus, Halocafeteria, Halospirulina, Hantzschia, Hymenomonas, Isochrysis, Lepocinclis, Limnothrix, Micractinium, Microactinium, Microcystis, Monochrysis, Monodus, Monoraphidium, Muriellopsis, Nannochloris, Nannochloropsis, Navicula, Neochloris, Neospongiococcum, Nephrochloris, Nephroselmis, Nitzschia, Nodularia, Nostoc, Ochromonas, Oedogonium, Oocystis, Oscillatoria, Ostreococcus, Parachlorella, Pavlova, Peridinium, Phaeodactylum, Picochlorum, Platymonas, Pleurochrysis, Pleurococcus, Porphyra, Porphyridium, Prochlorococcus, Prototheca, Prymnesium, Pseudanabaena, Pseudochlorella, Pseudochoricystis, Pseudoneochloris, Pyramimonas, Pyrobotrys, Rhodomonas, Scenedesmus, Schizochytrium, Scytonema, Skeletonema, Spirogyra, Stichococcus, Synechococcus, Tetrachlorella, Tetradesmus, Tetraselmis, Thalassiosira, Tisochrysis, Tolypothrix, Tribonema, Trichodesmium, Ulothrix, Vaucheria, Viridiella, Volvox, and genetically-engineered varieties or combinations (mixtures, mixed cultures, co-cultures or synthetic co-cultures) thereof.

In exemplary embodiments, the aquaculture medium including microorganisms capable of phototaxis contains at least one or more of the following groups of microorganisms (e.g. algae or microalgae) capable of phototaxis: Anabaena, Ankistrodesmus falcatus, Arthrospira (Spirulina) obliquus, Arthrospira (Spirulina) platensis, Botryococcus braunii, Chaetoceros gracilis, Chlamydomonas reinhardtii, Chlorella vulgaris, Chlorella pyrenoidosa, Chlorococcum littorale, Cyclotella cryptica, Dunaliella bardawil, Dunaliella salina, Dunaliella kone, Dunaliella tertiolecta, Dunaliella viridis, Euglena gracilis, Haematococcus pluvialis, Isochrysis galbana, Nannochloris, Nannochloropsis salina, Navicula saprophila, Neochloris oleoabundans, Nitzschia laevis, Nitzschia alba, Nitzschia communis, Nitzschia paleacea, Nitzschia closterium, Nostoc commune, Nostoc flagellaforme, Pleurochrysis carterae, Porphyridium cruentum, Prymnesium, Pseudochoricystis ellipsoidea, Scenedesmus obliquus, Scenedesmus quadricauda, Scenedesmus acutus, Scenedesmus dimorphus, Skeletonema costatum, Spirogyra, Spirulina, Synechoccus, Amphora, Fragilaria, Schizochytrium, Rhodomonas, and genetically-engineered varieties or combinations thereof. In an exemplary embodiment the algae or microalgae is selected from the group including or consisting of Dunaliella sp., Dunaliella bardawil, Dunaliella kone, Dunaliella salina, Dunaliella bioculata, Dunaliella granulata, Dunaliella maritima, Dunaliella minuta, Dunaliella parva, Dunaliella peircei, Dunaliella polymorpha, Dunaliella primolecta, Dunaliella pseudosalina, Dunaliella quartolecta, Dunaliella terricola, Dunaliella tertiolecta, and Dunaliella viridis.

In exemplary embodiments, the one or more algae or microalgae present in the microorganisms are selected from the group including or consisting of Dunaliella sp., Dunaliella bardawil, Dunaliella salina, Dunaliella kone, Dunaliella tertiolecta, Dunaliella parva and Dunaliella viridis, and any other combination thereof. In exemplary embodiments, the algae or microalgae is Dunaliella salina, Dunaliella bardawil, or a combination thereof.

In exemplary embodiments, the algae includes or consists of microalgae. In an exemplary embodiment, the algae or microalgae have not been genetically modified or do not originate from genetically engineered algae or microalgae.

In exemplary embodiments, the aquaculture medium including microorganisms capable of phototaxis contains at least one population of algal species with flagella, cilia and/or photosensitive eyespots. Flagella are a tail-like projection that protrudes from the cell body of certain algae and functions in locomotion. Cilia are an adaptation that allows independent cellular creatures, like algae, to move around in search of food. Photosensitive eyespots are found in some free-swimming unicellular algae. Photosensitive eyespots are sensitive to light. They enable the algae to move in relation to a light source. Such algae have the capability of independent motion, phototaxis, and can move towards the surface during daylight. Phototaxis is the movement of microalgae in response to light. For example, certain algae (e.g., Dunaliella) can perceive light by means of a sensitive eyespot and move to regions of higher light concentration to enhance photosynthesis.

In exemplary embodiments, the aquaculture medium and/or algal aquaculture zone including microorganisms capable of phototaxis contains microorganism capable of growing at elevated salinity, for example, a medium having a salinity of about 5 wt % to about 27 wt %.

### Sources of Aquaculture Medium

Suitable sources of aquaculture medium, optionally containing microorganisms capable of phototaxis, can include, but are not limited to, tubular reactors, photobioreactors, enclosed raceways, covered ponds, open raceways, open ponds, earthen ponds, ponds in greenhouses, clear plastic bags hung either indoors or outdoors, fermenters, naturally occurring bodies of water, solar salt ponds, and combinations thereof. Any design of a fermenter, pond, or bioreactor known in the art for the growth of algal biomass is acceptable for providing aquaculture medium, optionally containing microorganisms capable of phototaxis, to the algal aquaculture zone. In some embodiments, the photobioreactors rely on solar radiation for light, while others utilize man-made lights or solar collectors that channel solar radiation to the photobioreactor. In other embodiments, aquaculture medium, optionally containing microorganisms capable of phototaxis, may be derived from fermenters where they are grown on sugars, cellulose, and/or other biomass. Either phototrophic or heterotrophic algae grown in one or more bioreactors may be used as a source of aquaculture medium, optionally containing microorganisms capable of phototaxis, in the processes and systems disclosed herein.

Suitable tubular reactors include those constructed from glass; or from plastics, including, but not limited to polyethylene, polypropylene, polycarbonate, acrylic, polyesters, specialty polyesters, for example, cyclohexanedimethanol modified polyesters and Tritan® from Eastman Chemical Company, and combinations thereof. The tubular photobioreactors may be constructed so that they are rigid in nature, for example, those constructed from glass, polyester, or polycarbonate.

Another suitable source of aquaculture medium, optionally containing microorganisms capable of phototaxis, is enclosed raceways, for example, racetrack-shaped ponds that are covered to protect the microorganisms from inclement weather, and they have the ability to control the degree of agitation via a mixing device. These enclosed raceways are similar to an algal pond located in a greenhouse. This type of aquaculture tends to be expensive, but may be preferred at latitudes greater than about 35 degrees where the winter temperatures are too low to support efficient algal growth.

Another suitable source of aquaculture medium, optionally containing microorganisms capable of phototaxis, is fermenters, which are those used for the production of non-photosynthetic organisms. Fermenters are especially useful for growing genetically modified algae that must be contained. Other algae that are grown to relatively high concentration in fermenters are those that require sugars or a carbon source, for example, sugar that is not directly carbon dioxide. Specifically, fermenters are preferred when heterotrophic algae are utilized.

Suitable open ponds for sourcing aquaculture medium, optionally containing microorganisms capable of phototaxis, include those used for growing shrimp, fish, shellfish, or other types of marine organisms, or combinations thereof. Other suitable open ponds include those that are used for the production of solar salt or other minerals. Open ponds may either be lined or unlined, although the latter provides advantages from an economic standpoint. The open ponds may be lined with plastic or bentonite or clay or other material that is impervious to the flow of water, or combinations thereof. Pond liners constructed from various plastics including, but not limited to polyethylene, polypropylene, vinyl, and combinations thereof may be used. Bentonite, clay, salt, and other minerals and combinations thereof may also be useful to reduce or minimize leakage of the algal growth medium into the environment.

Enclosed photobioreactors can also be used as a source of aquaculture medium, optionally containing microorganisms capable of phototaxis, in the processes and systems disclosed herein. These photobioreactors are transparent so that the microorganisms they contain can utilize the sunlight. These photobioreactors have also been proposed for use in the production of biofuels, nutraceuticals and specialty oils. These enclosed photobioreactors may include plastic bags, glass and plastic tubes, ponds in green-house structures, and the like. Tubular reactors were popularized by GreenFuel Technologies Corporation of Cambridge, Massachusetts for the production of biofuels, but the technology was economically unsuccessful. Plastic bag bioreactors are typified by those utilized by Algenol Biofuels of Fort Myers, Florida. Fermenters have been used by Solazyme for the production of specialty oils and nutraceuticals. Martek has also used fermenters for the production of nutraceuticals. Although the capital cost of constructing a bioreactor from plastic instead of steel is substantially reduced, a plastic bioreactor is still so expensive that one commercial application to date is for the production of astaxanthin, a carotenoid, which is a high-value product. Thus, the use of the enclosed photobioreactors as a source of aquaculture medium, optionally containing microorganisms capable of phototaxis, can be included within the processes and systems disclosed herein.

Open-pond bioreactors can also be used as a source of aquaculture medium, optionally containing microorganisms capable of phototaxis, in the processes and systems disclosed herein. These bioreactors are generally classified as natural, intensive, and extensive. The natural open-pond bioreactors are defined as those naturally occurring ponds where the conditions are right to grow algae. These ponds may contain either fresh or saline water, and they are unmanaged in terms that they lack controlled fertilizer addition and mechanical agitation. Natural open ponds that contain algae are common along the shores of the Great Salt Lake in Utah.

Both the intensive and extensive modes of open-pond bioreactors require the controlled addition of fertilizers to the medium in order to supply the necessary nutrients, for example, phosphorus, nitrogen, iron, and trace metals, that are necessary for biomass production through photosynthesis. The primary difference between the two modes of production is mixing of the growth medium. Intensive open-pond bioreactors employ mechanical mixing devices while extensive open-pond bioreactors rely on happenstance mixing. Therefore, factors that affect microorganism growth can be more accurately controlled in intensive open-pond bioreactors.

Intensive open-pond bioreactors are frequently constructed by erecting a concrete block perimeter that is used to contain a plastic liner. Brine depth generally is controlled at about 20 centimeters, which has been considered to be the optimum depth for producing algal biomass. A number of configurations of these ponds have been proposed. However, the open air raceway ponds are important commercially. Raceway ponds employ paddle wheels to provide mixing. Chemical and biological parameters are carefully controlled, including salt and fertilizer concentrations, pH of the brine, and purity of the culture.

Extensive open-pond bioreactors has been practiced in the hot and arid regions of Australia for the production of beta-carotene. Outdoor ponds for extensive aquaculture generally are larger than those for intensive aquaculture and normally are constructed in lake beds. The open air ponds are bounded by earthen dikes. No mechanical mixing devices are employed.

Algal bioreactors that utilize these types of aquaculture systems, others know in the art, and combinations thereof, may be used to supply aquaculture medium, optionally containing microorganisms capable of phototaxis, to the algal to the aquaculture zones.

### Algal Aquaculture Zone

In exemplary embodiments, the algal aquaculture zone includes an open pond.

In exemplary embodiments, the agal aquaculture zone contains at least one open pond, closed pond, raceway pond, tubular reactor, photobioreactor, enclosed raceway, covered pond, open raceway pond, earthen pond, pond in a greenhouse, fermenter, naturally occurring body of water, solar salt pond, or any combinations thereof.

In exemplary embodiments, algae or microalgae from divisions Bacillariophyta, Chlorophycophyta, Chrysophycophyta, Cyanophycophyta, Cryptophycophyta, Phaeophycophyta, Pyrrhophycophyta, Rhodophycophyta or combinations thereof are suitable for use in the present disclosure.

The algae or microalgae used can include, but are not limited to, one or more species from the following genera: Acutodesmus, Achnahtes, Amphipora, Amphora, Anabaena, Ankistrodesmus, Arthrospira (also known as Spirulina), Asteromonas, Asterionella, Boekelovia, Borodinella, Botryococcus, Bracteacoccus, Carteria, Chaetoceros, Chlamydomonas, Chlorella, Chlorococcum, Chlorogonium, Chloromonas, Chroomonas, Chrysophaera, Ceratium, Closterium, Coccolithus, Coelastrella, Coscinodiscus, Cosmarium, Cricosphaera, Crocosphaera, Crypthecodinium, Cryptomonas, Cyanocystis, Cyanospira, Cyclotella, Desmodesmus, Ditylum, Dunaliella, Ellipsoidon, Emiliania, Eremosphaera, Euglena, Fragilaria, Franceia, Galdieria, Gracilaria, Graesiella, Guinardia, Haematococcus, Halocafeteria, Halospirulina, Hantzschia, Hymenomonas, Isochrysis, Lepocinclis, Limnothrix, Micractinium, Microactinium, Microcystis, Monochrysis, Monodus, Monoraphidium, Muriellopsis, Nannochloris, Nannochloropsis, Navicula, Neochloris, Neospongiococcum, Nephrochloris, Nephroselmis, Nitzschia, Nodularia, Nostoc, Ochromonas, Oedogonium, Oocystis, Oscillatoria, Ostreococcus, Parachlorella, Pavlova, Peridinium, Phaeodactylum, Picochlorum, Platymonas, Pleurochrysis, Pleurococcus, Porphyra, Porphyridium, Prochlorococcus, Prototheca, Prymnesium, Pseudanabaena, Pseudochlorella, Pseudochoricystis, Pseudoneochloris, Pyramimonas, Pyrobotrys, Rhodomonas, Scenedesmus, Schizochytrium, Scytonema, Skeletonema, Spirogyra, Stichococcus, Synechococcus, Tetrachlorella, Tetradesmus, Tetraselmis, Thalassiosira, Tisochrysis, Tolypothrix, Tribonema, Trichodesmium, Ulothrix, Vaucheria, Viridiella, Volvox, and genetically-engineered varieties or combinations (mixtures, mixed cultures, co-cultures or synthetic co-cultures) thereof.

In exemplary embodiments, the one or more algal aquaculture zones contain microorganisms capable of phototaxis. These microorganisms can include species selected from Anabaena, Ankistrodesmus falcatus, Arthrospira (Spirulina) obliquus, Arthrospira (Spirulina) platensis, Botryococcus braunii, Chaetoceros gracilis, Chlamydomonas reinhardtii, Chlorella vulgaris, Chlorella pyrenoidosa, Chlorococcum littorale, Cyclotella cryptica, Dunaliella bardawil, Dunaliella salina, Dunaliella tertiolecta, Dunaliella viridis, Euglena gracilis, Haematococcus pluvialis, Isochrysis galbana, Nannochloris, Nannochloropsis salina, Navicula saprophila, Neochloris oleoabundans, Nitzschia laevis, Nitzschia alba, Nitzschia communis, Nitzschia paleacea, Nitzschia closterium, Nostoc commune, Nostoc flagellaforme, Pleurochrysis carterae, Porphyridium cruentum, Prymnesium, Pseudochoricystis ellipsoidea, Scenedesmus obliquus, Scenedesmus quadricauda, Scenedesmus acutus, Scenedesmus dimorphus, Skeletonema costatum, Spirogyra, Spirulina, Synechoccus, Amphora, Fragilaria, Schizochytrium, Rhodomonas, and genetically-engineered varieties or combinations thereof. In an exemplary embodiment the algae or microalgae is selected from the group including or consisting of Dunaliella sp., Dunaliella bardawil, Dunaliella kone, Dunaliella salina, Dunaliella bioculata, Dunaliella granulata, Dunaliella maritima, Dunaliella minuta, Dunaliella parva, Dunaliella peircei, Dunaliella polymorpha, Dunaliella primolecta, Dunaliella pseudosalina, Dunaliella quartolecta, Dunaliella terricola, Dunaliella tertiolecta, and Dunaliella viridis.

In exemplary embodiments, the one or more algae or microalgae present in the microorganisms are selected from the group including or consisting of Dunaliella sp., Dunaliella bardawil, Dunaliella salina, Dunaliella kone, Dunaliella tertiolecta, Dunaliella parva and Dunaliella viridis, and any combination thereof. In exemplary embodiments, the algae or microalgae is Dunaliella salina, Dunaliella bardawil, or a combination thereof.

In exemplary embodiments, the algae includes or consists of microalgae. In an exemplary embodiment, the algae or microalgae have not been genetically modified or do not originate from genetically engineered algae or microalgae.

In exemplary embodiments, the process for maintaining aerobic conditions and/or controlling algae age in the at least one algal aquaculture zone includes monitoring and/or measuring the dissolved oxygen content of the at least one algal aquaculture zone using a dissolved oxygen sensor. The dissolved oxygen sensor can be used to determine the optimum quantities of bacteria to be removed from the at least one algal aquaculture zone and/or the amount of microorganisms capable of phototaxis to be removed and/or recovered from the at least one algal aquaculture zone.

The dissolved oxygen sensor can monitor the aerobic conditions of the at least one algal aquaculture zone by any means employed within the art, for example, but not limited to, fluorescent-based oxygen sensing, galvanic and/or polarographic means. In some instances, these sensors can measure not only the dissolved oxygen but also oxygen that is above the solubility limit, i.e., supersaturated in oxygen. Throughout this document, we use the terms dissolved oxygen to correspond to measurements that one could obtain from a dissolved oxygen sensor, and thus could include supersaturated oxygen as well.

In exemplary embodiments, the dissolved oxygen sensor measures and/or monitors the aerobic condition of the at least one algal aquaculture zone by tracking the millivoltage produced from a fluorescent-based oxygen sensor, wherein the trend in millivoltage values is used to determine the quantity of bacteria and/or microorganisms capable of phototaxis to be removed and/or recovered from the at least one algal aquaculture zone.

In exemplary embodiments, the process for maintaining aerobic conditions and/or controlling the algae age in the at least one algal aquaculture zone includes monitoring the oxidation-reduction potential of the at least one algal aquaculture zone with a dissolved oxygen sensor, wherein the instant oxidation-reduction potential and/or the trend in oxidation-reduction potentials are used to determine the quantity of the bacteria and/or microorganisms capable of phototaxis to be removed and/or recovered from the at least one algal aquaculture zone.

In exemplary embodiments, the process for maintaining aerobic conditions and/or controlling the algae age in the at least one algal aquaculture zone includes maintaining the oxidation-reduction potential of the algal aquaculture zone at positive En values, where Eh is a measure of the redox (oxidation-reduction) state of solutes in a solution. Eh is a measurement of electrical potential and has the units of volts. Values of Eh range from −0.6 to +0.9 V, with a zero voltage characterizing a solution without a driving force to either oxidize or reduce. The maintenance of the oxidation-reduction potential of the at least one algal aquaculture zone can be achieved by determining the quantity of the bacteria and/or microorganisms capable of phototaxis to be removed and/or recovered from the at least one algal aquaculture zone.

In exemplary embodiments, the process for maintaining aerobic conditions and/or controlling the algae age in the at least one algal aquaculture zone includes maintaining oxidation-reduction potentials in the at least one algal aquaculture zone and/or the illumination zone at Eh values above 0 V.

In exemplary embodiments, the process for maintaining aerobic conditions and/or controlling the algae age in the at least one algal aquaculture zone includes determining the dissolved oxygen level in the at least one algal aquaculture zone, and optionally maintaining it above 0 mg/L.

In exemplary embodiments, the process for maintaining aerobic conditions and/or controlling the algae age in the at least one algal aquaculture zone includes regulating the quantity of algal biomass introduced into and removed from the algal aquaculture zone. For example, if the trend of the dissolved oxygen content at the lowest points at night is approaching zero mg O₂/liter, then additional algal biomass should be removed in order to control oxygen consumption in the at least one algal aquaculture zone. Alternatively, instead of removing algae and/or algal biomass from the upper layer containing microorganisms capable of phototaxis, a portion of the lower layer may be removed to remove bacteria and archaea from the at least one algal aquaculture zone.

In exemplary embodiments, the process for maintaining aerobic conditions and/or controlling the algae age in the at least one algal aquaculture zone includes modifying the flowrate ratio of an aqueous medium and the aquaculture medium into the at least one algal aquaculture zone.

The aqueous medium can have an elevated salinity, for example, a salinity of about 5 wt % to about 27 wt %. For example, the aquaculture medium can have a salinity that is about 5% or greater than 5%, about 6% or greater than 6%, about 7% or greater than 7%, at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, at least about 25 wt %, at least about 26 wt %, or at least about 27 wt %. In exemplary embodiments the aquaculture medium is saturated with salt. In other exemplary embodiments, the aquaculture medium can have a salinity from about 7 wt % to saturation, from about 10 wt % to saturation, from about 15 wt % to saturation, from about 20 wt % to saturation, from about 5 wt % to about 25 wt %, from about 5 wt % to about 20 wt %, from about 10 wt % to about 20 wt %, from about 5 wt % to about 15 wt %, from about 10 wt % to about 15 wt %, or from about 5 wt % to about 10 wt %.

The aqueous medium can include water and salts that are found in seawater, terminal lakes, or aquifers. The aqueous medium may include combinations of ions found in seawater. The concentration of the salts in the aqueous medium may range from just a trace to saturation so that suitable names to describe the salinity of the aqueous medium would range from fresh water, brackish water, salt water, brine, and saturated brine, respectively, as the salts in the aqueous medium increase. The desired concentration of the salts in the aqueous medium will depend on the type of algae that are being grown in the algal aquaculture zone and cover the range from fresh water to saturated brine.

Various combinations of ions found in seawater may be included within the aqueous medium, for example, ion combinations derived from one or more of the following sources including: water derived from streams, lakes, rivers, or other sources associated with fresh water; water derived from underground aquifers that may include various ion concentrations; water derived from industrial, agricultural, or municipal sources that may or may not have received treatment; or water derived from brackish sources where fresh water is combined with sea water or ocean water in various proportions; sea water or ocean water that may be derived from the various seas and oceans located around the globe; or combinations thereof. The combination of ions for the aqueous medium may be derived directly from these sources or may be derived by evaporating the desired amount of water from any of these sources to leave the desired ion-rich solution for use in the aqueous medium.

The ion combination that can be included with the aqueous medium may differ for different algae, an example of which is disclosed in U.S. Pat. No. 6,986,323, the contents of which are incorporated herein by reference in their entirety. Combination of ions can be obtained by the evaporation of sea water or ocean water, including the evaporation of ancient sea waters that form terminal lakes, for example, the Great Salt Lake in Utah and that form various aquifers. The combination of ions can also be derived from crystallizers, for example, those wherein sodium chloride ions are precipitated.

### Illumination Zone

The process 101 includes exposing the aquaculture medium including microorganisms capable of phototaxis to at least one light source in the at least one aquaculture zone, the light source creating an illumination zone in the at least one algal aquaculture zone.

The process 101 depicted in FIG. 1 also includes allowing the microorganisms capable of phototaxis, contained within the aquaculture medium and exposed to the at least one light source, to move toward the at least one light source; thereby forming an upper layer containing the microorganisms capable of phototaxis and a lower layer in the illumination zone.

The illumination zone can have, at least partly, a width to depth ratio of about 100:0.5 to 1:1. In exemplary embodiments, at least part of the illumination zone or the entirety of the illumination zone has a width to depth ratio from about 19:0.5 to about 38:1. In other exemplary embodiments, at least part of the illumination zone or the entirety of the illumination zone has a width to depth ratio from about 50:0.5 to 1:0.5.

In exemplary embodiments, the process for maintaining aerobic conditions and/or controlling algae age in the at least one algal aquaculture zone includes monitoring the dissolved oxygen content of the illumination zone using a dissolved oxygen sensor. The dissolved oxygen sensor can monitor the aerobic conditions of the illumination zone by any means employed within the art, for example, but not limited to, fluorescent-based oxygen sensing, galvanic and/or polarographic means. In some instances, these sensors can measure not only the dissolved oxygen but also oxygen that is above the solubility limit, i.e., supersaturated in oxygen. Methods and equipment to measure DO have been published by Mettler-Toledo in their booklet entitled Hints and Tips to measure Dissolved Oxygen and pH″, Hanna Instruments has also published a description of these methods and supply equipment for the measurement of dissolved oxygen. The polarographic method works well at salinities less than seawater. The galvanic method was shown to work at elevated salt levels, but long term use may lead to corrosion of the galvanic cell. The LDO or luminescent probe worked well at all salinities and was the method used in the examples.

In exemplary embodiments, the dissolved oxygen sensor measures and/or monitors the aerobic condition of the illumination zone by tracking the millivoltage produced from a fluorescent-based oxygen sensor, wherein the trend in millivoltage values is used to determine the quantity of bacteria and/or microorganisms capable of phototaxis to be removed and/or recovered from the illumination zone.

In exemplary embodiments, the process for maintaining aerobic conditions and/or controlling the algae age in the at least one algal aquaculture zone includes monitoring the oxidation-reduction potential of the illumination zone with a dissolved oxygen sensor, wherein the instant oxidation-reduction potential and/or the trend in oxidation-reduction potentials are used to determine the quantity of the bacteria and/or microorganisms capable of phototaxis to be removed and/or recovered from the illumination zone.

In exemplary embodiments, the process for maintaining aerobic conditions and/or controlling the algae age in the at least one algal aquaculture zone includes maintaining the oxidation-reduction potential of the illumination zone at positive En values, where Eh is a measure of the redox (oxidation-reduction) state of solutes in a solution. The maintenance of the oxidation-reduction potential of the illumination zone can be achieved by determining the quantity of the bacteria and/or microorganisms capable of phototaxis to be removed and/or recovered from the illumination zone.

In exemplary embodiments, the process for maintaining aerobic conditions and/or controlling the algae age in the illumination zone includes determining the dissolved oxygen level in the illumination zone, and optionally maintaining it above 0 mg/L.

In exemplary embodiments, the exposure of the aquaculture medium including microorganisms capable of phototaxis to at least one light source in at least one algal aquaculture zone occurs from about 4 hours to about 24 hours a day, from about 5 hours to about 24 hours a day, from about 6 hours to about 24 hours a day, from about 7 hours to about 24 hours a day, from about 8 hours to about 24 hours a day, from about 9 hours to about 24 hours a day, from about 10 hours to about 24 hours a day or any amount of time ranging from 4 hours to 24 hours. In an exemplary embodiment the exposure to at least one light source occurs within a time, e.g., 4 hours to 10 hours, from the sunrise to the sunset or from the sunset to the sunrise (i.e., during night).

In exemplary embodiments, the aquaculture medium including microorganisms capable of phototaxis is exposed to two or more light sources (e.g. at least the sun and an artificial light, or at least two artificial lights) in the illumination zone.

The one or more light sources can include, but are not limited to, any source that provides solar radiation, for example, the sun, and/or artificial light, for example, LED lights, Halogen lamps, incandescent lights, fluorescent lights, and combinations thereof. In one exemplary embodiment the key aspect of the light source is that it induces the phototaxis response in the algae or microalgae that induces them to approach the gas-liquid interface in the illumination zone. The light source can be elevated above a weir or above a skimming unit so that the illumination zone extends so that the phototaxic organism can move from the bulk of the aquaculture zone to the upper layer containing microorganisms. In one embodiment, the light source would emit the wavelength to which the phototaxic organism responds.

Without being bound to any theory, the concentration of the microorganisms capable of phototaxis at the surface of the aquaculture medium offers the opportunity for the selective removal of the microorganisms from the bacteria that remain more uniformly distributed throughout the medium.

In exemplary embodiments, the upper surface layer is located within the illumination zone and/or at least partly in the upper surface of the aquaculture medium in the at least one algal aquaculture zone not exposed to the at least one light source.

The concentration of microorganisms capable of phototaxis in the upper layer can range from about 10,000 cells per milliliter to about 2,000,000 cells per milliliter, depending on the concentration of the microorganism in the algal aquaculture zone and on the thickness of the upper layer being considered. Specifically, phototaxic microalgae may accumulate at high concentrations in the top centimeter of the aquaculture medium in the illumination zone. In this case, it may be desirable to skim just the top one centimeter upper layer containing microorganism to send the highest concentration to the harvesting zone.

In exemplary embodiments, the upper layer containing the microorganisms is 1 to 100 mm (millimeters) deep, 1 to 75 mm deep, 1 to 50 mm deep, 1 to 25 mm deep, and 1 to 10 mm deep.

In exemplary embodiments, the lower layer contains microorganisms incapable of phototaxis.

In exemplary embodiments, the lower layer is at least 10 mm below the upper surface of the aquaculture medium, at least 25 mm below the upper surface of the aquaculture medium, at least 50 mm below the upper surface of the aquaculture medium, at least 75 mm below the upper surface of the aquaculture medium, or at least 100 mm below the upper surface of the aquaculture medium.

In exemplary embodiments, the process for maintaining aerobic conditions and/or controlling the algae age in the at least one algal aquaculture zone includes separating at least part of the upper layer containing the microorganisms capable of phototaxis and any biomass produced from those microorganisms from at least part of the lower layer by transferring at least part of the upper layer to a filtration unit containing a filter. The filtration unit can be configured to selectively remove the microorganisms capable of phototaxis and any additional biomass produced by those microorganisms from the aquaculture medium and at least some of the bacteria. The filtration unit can be configured to transport the filtered microorganisms capable of phototaxis and any additional biomass to a harvesting zone.

In exemplary embodiments, the process for maintaining aerobic conditions and/or controlling the algae age in the at least one algal aquaculture zone includes separating at least part of the upper layer containing the microorganisms capable of phototaxis and any biomass produced from those microorganisms from at least part of the lower layer by transferring at least part of the upper layer to a sedimentation unit. The sedimentation unit can be configured to allow the microorganisms capable of phototaxis and any biomass produced from those microorganisms that rose to the upper layer in the at least one aquaculture zone to separate from the bacteria and aquaculture medium while in the sedimentation unit, thereby separating the microorganisms capable of phototaxis and any additional biomass from the aquaculture medium and at least some of the bacteria. The microorganisms capable of phototaxis and any additional biomass produced from those microorganisms may either settle to the top or bottom of the sedimentation unit. The sedimentation unit can also be configured to transport the sedimented microorganisms capable of phototaxis and any additional biomass produced from those microorganisms to a harvesting zone. The sedimentation unit can also be configured to recycle at least some of the aquaculture medium present in the transferred upper layer back to the at least one algal aquaculture zone.

In exemplary embodiments, the process for maintaining aerobic conditions and/or controlling the algae age in the at least one algal aquaculture zone includes removing at least part of the lower layer containing microorganisms incapable of phototaxis from the at least one algal aquaculture zone by transferring at least part of the lower layer to a separation zone and/or to a drainage pipe. In exemplary embodiments, transferring of at least part of the lower layer to the separation zone and/or the drainage pipe is controlled by an underflow weir. In an exemplary embodiment, the lower layer from the drainage pipe may be recycled as aquaculture media or treated to reduce the concentration of the microorganism incapable of phototaxis.

In exemplary embodiments, the process for maintaining aerobic conditions and/or controlling the algae age in the at least one algal aquaculture zone includes skimming at least part of the upper surface over at least one overflow weir into a separation zone. The at least one overflow weir can be made of materials including, but not limited to wood, cement, plastic, steel, or combinations thereof.

### Separation Zone

The process 101 depicted in FIG. 1 includes separating at least part of the upper layer containing the microorganisms capable of phototaxis from at least part of the lower layer by selectively transferring at least part of the upper layer into a separation zone. The process 101 depicted in FIG. 1 also includes separating at least part of the lower layer from the upper layer by removing at least part of the lower layer from the bottom of the at least one algal aquaculture zone.

In exemplary embodiments, the illumination zone is located next to the separation zone. In that case, the algae capable of phototaxis rise to the upper layer so that they can be separated from the lower layer by a separation step or device, such as a skimmer or an overflow weir. In an exemplary embodiment, the size of the illumination zone should provide the organisms capable of phototaxis ample residence time to move to the upper layer so that the amount or volume of the upper layer containing the microorganisms can be controlled in order to control the overall flowrate going to the separation zone. For example, if a skimmer or overflow weir with a limited lip length is located at the at least one algal aquaculture zone's border, a semi-circular illumination zone would offer similar residence time in the illumination zone so that phototaxis could occur regardless of which direction the streamlines approach the skimmer or overflow weir. If a circular skimmer is located in the middle of at least one algal aquaculture zone, then a circular illumination zone would be optimal in order to allow sufficient residence time for the phototaxic microorganisms to accumulate in the upper layer containing microorganisms before they enter the circular separation zone. The removal of some portion of the upper layer containing microorganisms results in the flow of the upper layer towards the lip of an overflow weir or skimmer, thus continuously moving the upper layer through the illumination zone into the separation zone.

In exemplary embodiments, a transition zone is located between the illumination zone and the separation zone. The transition zone can be located at least partly in the separation zone and/or can be located at least partly in the illumination zone. In exemplary embodiments, at least part of the overflow weir or skimmer is located in the transition zone.

In exemplary embodiments, the transition zone is a canal that optionally possesses the width to depth ratio essentially constant.

In exemplary embodiments, the process for maintaining aerobic conditions and/or controlling algae age in the at least one algal aquaculture zone includes determining oxidation-reduction potentials in the at least one algal aquaculture zone and/or the illumination zone before separating at least part of the upper surface layer from at least the lower layer in the at least one algal aquaculture zone, illumination zone, and/or the transition zone into the separation zone.

In exemplary embodiments, the at least one algal aquaculture zone and/or the illumination zone can include a dished area. The at least one algal aquaculture zone and/or the illumination zone can have their depths increased so that the liquid depth in the dished area ranges between about 1.5 to 5 times the mean liquid depth in the algal aquaculture zone. By increasing the depth in the illumination zone and/or the at least one algal aquaculture zone, wind mixing of the aquaculture medium is reduced in the dished area and this allows the phototaxic microorganisms to better rise to the surface that is illuminated by the lighting system, without being mixed by currents generated by the wind. The size of the dished area can be designed so that the phototaxic microorganisms have sufficient time to move into the upper layer, so they can be separated from the microorganisms incapable of phototaxis in the lower layer.

The separation zone may contain any method or device known in the art to separate an upper liquid layer from a lower liquid layer. Suitable devices include, but are not limited to skimmers, overflow weirs, underflow weirs, sedimentation units, filtration units, other methods commonly employed in the art, and/or combinations thereof.

The one or more overflow and/or underflow weirs can be constructed of wood, concrete, plastic, steel, cement or combinations thereof. In one embodiment, the weir structure is constructed of cement or concrete in a manner where there are vertical slots for one or more weir boards that can be inserted into the slots to control the liquid level in the aquaculture zone while allowing hydraulic flow of the aquaculture medium through the weir structure. The cement or concrete structure may also have a moving gate that controls the flow of the aquaculture medium through the weir. Likewise, a steel structure can be used to hold the weir boards or movable gate.

The one or more skimmers can be any of those provided commercially, or they can be constructed independent of a vendor. Examples of skimmers that could be used in this application include the following designs of floating skimmers that are similar to pool skimmers; floating rotating disc skimmers; floating drum skimmer, floating weir skimmer; and other types of skimmers used in the art, and combinations thereof.

In exemplary embodiments, at least part of the upper layer containing the microorganisms capable of phototaxis is separated from at least a part of the lower layer containing the bacteria. The bottom layer may be selectively removed by a drain system at the bottom of the at least one algal aquaculture zone.

### The Harvesting Zone

The process 101 depicted in FIG. 1 includes transferring at least part of the microorganisms capable of phototaxis of the upper layer from the separation zone to a harvesting zone.

In exemplary embodiments, the transferring of at least part of the microorganisms capable of phototaxis of the upper layer from the separation zone to a harvesting zone includes pumping the microorganisms capable of phototaxis and any biomass produced by those microorganisms from the separation zone to the harvesting zone.

Microorganisms capable of phototaxis and any biomass produced by those microorganisms recovered from the algal aquaculture zone can be transported to a harvesting zone for additional processing. The harvesting zone can include one or more of an adsorptive bubble separation unit, a centrifugation unit, a flocculation unit, a sedimentation unit, a filtration unit, and/or combinations thereof.

In exemplary embodiments, the process for maintaining aerobic conditions and/or controlling the algae age in the at least one algal aquaculture zone includes harvesting at least part of the microorganisms capable of phototaxis from the upper surface layer of the at least one aquacultures zone in the harvesting zone. The harvesting can include at least one or more of rupturing the microorganisms capable of phototaxis, performing adsorptive bubble separation, centrifuging the microorganisms or ruptured microorganisms, flocculating the microorganisms or ruptured microorganisms, sedimenting the microorganisms or ruptured microorganisms, filtering the microorganisms or ruptured microorganisms, and/or any combination thereof. The above processes can also be performed to harvest any biomass produced by the microorganisms capable of phototaxis that has been transferred to the harvesting zone.

Prior to an adsorptive bubble separation process, the microalgae may be conditioned for a number of reasons, including but not limited to increasing the performance of an algal dewatering unit (e.g., an adsorptive bubble separation unit). Suitable conditioning processes that may optionally be used prior to an adsorptive bubble separation unit include, but are not limited to adding a flotation aid, adding a frother, adding a collector, adding an activator, and combinations thereof. Suitable conditioning processes result in conditioned microalgae.

In many adsorptive bubble separation processes, it is necessary to add reagents, known as collectors, which selectively render one or more of the species of particles in the feed hydrophobic, thereby assisting in the process of collection by the gas bubbles. Activators aid the adsorption of the collector to certain particles increasing the number of those particles which become hydrophobic. Depressors inhibit the adsorption of the collector to certain undesired particles, decreasing the number of those particles which become hydrophobic. Also, frothing agents or frothers may be added to assist in the formation of a stable froth on the surface of the liquid. The process of admitting these various flotation chemicals to the system is known as conditioning.

Sedimentation may be used as a conditioning process, as some algae may be flocculated or separated by placing the microalgae in a quiescent zone. Some algae, for example Haematococcus pluvialis can be separated from the growth medium by sedimentation resulting from the addition of alum or the lack of agitation of the growth medium. For example, U.S. Pat. No. 5,541,056, the contents of which are incorporated herein by reference in their entirety, teaches a process of concentrating Haematococcus pluvialis by sedimentation. Other algae, for example, Dunaliella salina can be flocculated and then separated by sedimentation. In this case, the addition of ferric chloride causes the flocculation. Any polymer or ions that cause flocculation may be used in this step. Cyclones can also be used to accelerate the rate of sedimentation. Any sedimentation equipment known in the art may be used to separate the flocculated algae from the growth medium prior to the adsorptive bubble separation unit.

Adsorption can be used as a conditioning process for algae in order to reduce the volumetric flow to the adsorptive bubble separation unit. Some algae, for example Dunaliella salina, can be concentrated by adsorbing the algae onto a hydrophobic surface, and then desorbing the algae with another fluid, as disclosed in U.S. Pat. No. 4,554,390, the contents of which are incorporated herein by reference in their entirety. Thus, adsorption may be used to preconcentrate the algae. A potential weakness with adsorption is the requirement of desorption with an aqueous phase that contains lower salinity brine than the growth medium. Obtaining less saline water is often difficult, and it dilutes the algal concentrate, thus making all of the downstream processing equipment larger.

Centrifugation is another suitable conditioning process for algae, and it may be combined with other processes, for example, sedimentation as taught in U.S. Pat. No. 4,115,949, the contents of which are incorporated herein by reference in their entirety. Sedimentation and centrifugation may be used either individually or in combination in order to generate an aqueous suspension of microalgae. Centrifuges known in the art may be used to affect algal preconcentration, as long as they can readily handle solids. Suitable centrifugation equipment can also be constructed from acceptable materials of construction in order to handle the ions present in the algal aquaculture media. When the algal culture media includes sodium chloride, stainless steel and/or plastic wetted parts on the centrifuge are present for robust operations. However, it should be noted that stainless steel is susceptible to stress corrosion cracking in a chloride environment. Suitable centrifuges known in the art include, but are not limited to those produced by Westfalia, Robatel, Bird, and Alfa Laval. Disc-stack centrifuges produced by Westfalia and Alfa Laval can also be used.

Deep bed filtration is another suitable conditioning process for algae, for example, those disclosed in U.S. Pat. No. 5,951,875, the contents of which are incorporated herein by reference in their entirety. Deep bed filtration may be useful to pre-concentrate the algal suspension prior to adsorptive bubble separation. Deep bed filtration relies upon a bed of granular media, usually sand, through which the growth medium containing algae flows downward under gravity. The algae are deposited in the pores of the granular media and in the interstitial spaces between the grains of media. Deep bed filtration should not be confused with straining filtration. Straining takes place on the surface of a mesh or fabric, and is only suitable to pre-concentrate algae that will not blind the filtration equipment. However, deep bed filters retain particles throughout their volume, with each pore and void space having a probability of retaining algal cells from the suspension that is flowing through. Suitable deep bed filtration media include those used in commercial processes, for example, quartz sand, garnet sand, anthracite, fiberglass, and mixtures thereof.

For adsorptive bubble processes to separate components, hydrophobic materials often include the component to be separated. Rendering material hydrophobic is another suitable conditioning process, wherein particle surfaces are treated with chemicals, or other techniques that selectively modify the component to be separated. In most cases, the particles are not initially hydrophobic, and the particles to be separated, harvested, or dewatered are made hydrophobic so they may be collected and separated with an adsorptive bubble separation unit. In other cases, the particles are all hydrophobic, and one component is modified to make it hydrophilic in order to keep it in the aqueous stream.

The surface of microalgae can be hydrophilic, therefore adsorptive bubble separation is less effective on whole, live microalgae cells without the addition of frothers and collectors. Often in order to use adsorptive bubble separation for dewatering microalgae, the microalgae cells must be conditioned to make them hydrophobic.

An example of the use of flocculation conditioning followed by adsorptive bubble separation to harvest microalgae was disclosed in U.S. Pat. No. 4,680,314, the contents of which are incorporated herein by reference in their entirety. U.S. Pat. No. 6,524,486, the contents of which are incorporated herein by reference in their entirety, also utilizes a flocculating agent to cause accumulations of microalgae that are then floated out using an adsorptive bubble process. This process requires the addition of flocculating agents, which are expensive, may have environmental concerns, and can contaminate the product or the growth medium.

A suitable conditioning process is to render the microalgae cells hydrophobic, to use adsorptive bubble separation, by “cell rupture” (also referred to as “cell disruption” or “lysis”). By rupturing the cell wall and/or cell membrane, lipids and other naturally hydrophobic components are exposed. These components and the cell fragments that are rendered hydrophobic can then be recovered with an adsorptive bubble separation process.

Cell rupture can be achieved by a number of processes which can be classified as chemical, physical or mechanical. The chemical processes include enzymatic digestion, detergent solubilization, lipid dissolution with a solvent, and alkali treatment (lipid saponification). Physical processes include osmotic shock, decompression, sonication, heat treatment, and freeze-thawing. Mechanical processes include grinding, high shear homogenization and pressure extrusion.

A common cell disruption process uses a pump to force the feed mixture at high pressure through a restricted orifice valve. Cell disruption may also be accomplished by three different mechanisms: impingement on the valve, high liquid shear in the orifice, and sudden pressure drop upon discharge, causing finally an explosion of the cell. As an example of this, the MICROFLUIDIZER™ cell disruption equipment of Microfluidics, Newton, MA, US utilizes pressures of about 5,000 to 40,000 psi (345-2760 bar). U.S. Pat. No. 6,405,948, the contents of which are incorporated herein by reference in their entirety, describes a process for liberating intracellular materials using a resonance disintegration mill in which a high speed rotor creates a series of compressions and decompressions.

U.S. Pat. Nos. 5,776,349 and 6,000,551, the contents of which are incorporated herein by reference in their entirety, disclose that microalgae cells are ruptured when the microalgal feed dispersion is subjected to a pressure drop created by pumping through an orifice. Pressure drops of 50 to 200 psig (3.4-14 bars) in combination with recirculation are claimed to render an acceptable percentage of the cells recoverable with an adsorptive bubble separation. However, it is expensive to pump the entire feed dispersion, where the growing media may represent greater than 99% of the mass, at these pressures to obtain a high percentage of cell rupture.

Another suitable conditioning process was disclosed in U.S. Pat. No. 8,512,998, the contents of which are incorporated herein by reference in their entirety. In this disclosure, a mill was used to rupture algae, and a preferred mode was a vibratory mill.

The use of any of the above processes to cause cell rupturing can also be used in the systems and processes disclosed herein.

Another suitable conditioning process is to render the microalgae cells hydrophobic by fracking the algae. The partial rupturing of algae is referred to as fracking. Fracked algae are preferable to completely ruptured algae due to the difference in size of the resulting particles. Particles resulting from fracking algae are larger than the particles resulting from the complete rupturing of algae and the adsorptive bubble separation process is more effective when dewatering the larger particles. Fracking the algae results in fracked algal cells having a hydrophobic location while retaining a significant portion of the intracellular material within the cellular membrane. This results in increased recovery of the intracellular material during the adsorptive bubble separation process. The intracellular material may include, but is not limited to algal oil, algal proteins, algal carotenoids, and combinations thereof. Fracking may take place in any device known in the art in which algae may be partially ruptured including but not limited to a vibratory mill, a French press, a pump, an agitated vessel, or combinations thereof.

The fracked microalgae can then be fed to an algal dewatering unit that may include any process of concentrating microalgae known in the art. Specific examples of technologies that are suitable for the algal dewatering unit include, but are not limited to adsorptive bubble separation processes, filtration, deep bed filtration, belt press, screw press, centrifugation, adsorption, sedimentation, and combinations thereof. An algal dewatering unit produces algal concentrate and a recycle tails from conditioned microalgae. The algal concentrate is concentrated in algal biomass relative to the conditioned microalgae. The recycle tails are depleted in algal biomass relative to the conditioned microalgae. The recycle tails may be recycled to the source of the algal aquaculture growth media, fed to a salt production process, or purged. Algal dewatering units may be used in series to produce algal concentrate. For example, fracked algal biomass may be passed through an adsorptive bubble separation process then passed through a centrifuge. The resulting algal concentrate being more concentrated than algal concentrate derived solely from an adsorptive bubble separation process. The goal of the algal dewatering unit is to increase the algal concentration in the algal concentrate prior to downstream processing.

In the adsorptive bubble separation process, as many physical stages or cells may be utilized in the form of a circuit as desired in order to provide optimum product recovery and concentration. These stages can be configured into a circuit pattern that is used in froth flotation that includes a rougher, one or more scavengers, and one or more cleaners.

The rougher functions as the initial adsorptive bubble separation stage where the aqueous suspension of microalgae from a source enters the adsorptive bubble separation unit. The objective of the rougher is to provide high recovery of the algal biomass. Adsorptive bubble separation equipment functioning as roughers can operate at higher superficial gas velocity and thinner froth depth than those functioning as concentrators or cleaners.

The resulting bubble and agglomerates of algal biomass float to the surface of the gas and liquid suspension where they collect as a concentrated froth. The concentrated froth overflows the rougher into a collection launder, and then into a holding tank. The contents from this tank are then charged to a first stage cleaner cell. The underflow from the rougher may be recycled to the rougher, or it is fed to the first scavenger stage to provide the desired recovery of the algal biomass.

The algal biomass exiting the rougher as the overflow froth can be concentrated by one or more cleaning stages. The purpose of the cleaning stages is to produce an algal concentrate stream that is suitable for downstream processing. The overflow from the cleaner closest to the rougher provides the feed for the next stage of cleaner, if one is used. The underflow from the cleaner can be recycled to the cleaner from whence it was derived or sent to the previous cleaner that is closer to the rougher or to the rougher. The froth in any stage of the circuit can be sprayed with wash water to reduce the concentration of salts, clay, or other undesired compounds, if desired.

The cleaning stages may include a single adsorptive bubble separation cell or may include multiple cells that receive a feed stream in series or in parallel. The underflow from the cleaners is either discarded, returned to the rougher, returned to the previous cleaner, passed to one or more scavengers arranged in series or parallel, or otherwise processed depending on the concentration of algal biomass in the underflow stream.

In the scavenging stages, algal biomass otherwise lost in the underflow from the rougher are collected and passed through another adsorptive bubble separation cell or multiple cells in order to maximize the recovery of the algal biomass. Concentrate from the scavenging stages is recycled to the cleaning stages, and the underflow from the scavenging stages is usually returned to the aquaculture system. The scavenging stages can include a single froth flotation vessel or can include multiple vessels that receive a feed stream in series or in parallel. The underflow from the rougher feeds the first scavenging stage, and multiple scavenging stages may be used in order to meet the desired algal biomass recovery.

Suitable adsorptive bubble separation equipment that can be used in the systems and processes described herein include, but are not limited to froth flotation, electrolytic flotation, foam fractionation, dissolved gas flotation, and combinations thereof.

Suitable adsorptive bubble separation technology for use in the systems and processes described herein can be based on the selective adsorption of algal cell material to the surfaces of gas bubbles passing through the algal suspension. Bubbles rise to form a froth that carries the algal material off overhead. Adsorptive bubble separation processes are suitable for removing a small mass of algae from a large volume of the aqueous suspension of microalgae. There are a variety of adsorptive bubble separation techniques, in some of which a froth is generated and in some of which no froth is generated. One useful adsorptive bubble separation technique for dewatering algae is a dispersed gas flotation technique termed “froth flotation”.

Suitable froth flotation devices for use as roughers, scavengers, or cleaners, include the commercially available equipment used for gas and liquid contact. These devices, which are also called “cells”, may be classified into two broad groups, mechanical and pneumatic flotation cells. The mechanical flotation cells can include a rotor and stator mechanism for dispersing the gas and providing efficient bubble and algae contact.

Pneumatic flotation cells can be most easily distinguished from mechanical flotation cells by the absence of a rotating impeller in the flotation device. In pneumatic flotation cells, bubble and algae collisions are produced by addition of gas only, without any moving parts. Pneumatic flotation cells may operate as roughers, cleaners, and scavengers and the operating conditions for each service will require slightly different operating parameters in order to optimize the flotation circuit.

Pneumatic and mechanical flotation cells may be used at any or all of the locations in a froth flotation circuit, depending on equipment performance and separation objectives. However, the pneumatic flotation cells can have advantages over mechanical cells. Higher recovery and throughput may be attained in a pneumatic device as compared to a mechanical device for a given equipment volume and energy input, which usually results in reduced capital and operating costs. Pneumatic devices can be produced from light weight, inexpensive plastics for cost savings and to promote mobility. These advantages and others are discussed below.

The mechanical and pneumatic flotation cells described herein can have several operating parameters in common, including the gas phase superficial velocity, Jg; the gas to feed ratio; the liquid residence time in the flotation device; flotation aid dosage; and the nature of the flotation gas. Several design parameters are also common to various froth flotation devices, including the aspect ratio of the collection zone; the aspect ratio of the separation zone; the process of phase contact, including cocurrent flow, countercurrent flow, crossflow, and mechanical mixing; the process of separating the bubble and algal agglomerates from the pulp; and the process of bubble generation.

Performance of the froth flotation device is quantified in terms of the algal biomass recovery, lipid recovery, and the concentration of salts, and clay in the clean algal biomass. There are several geometrical and operating parameters that are specific to each type of froth flotation device, but the major parameters named above are common to the entire field of flotation processes described herein.

Mechanical flotation cells can employ a rotor and stator mechanism for flotation gas induction, bubble generation, and liquid circulation providing for bubble and algal biomass collision. The ratio of vessel height to diameter, termed the “aspect ratio”, usually varies from about 0.7 to 2. Four or more cells, each having a centrally mounted rotor and stator mechanism, can be arranged in series to approach substantially perfect mixing and thereby to minimize liquid phase short circuiting. An auxiliary blower can be installed to provide sufficient gas flow to the cell. Mechanical cells may be sealed if desired to facilitate operation to control the flow of the flotation gas.

The flotation gas is dispersed into fine bubbles by a rotating impeller, which serves as the bubble generator. The rotating impeller creates a low pressure zone that induces gas to flow through an aspiration tube into the collection zone where it is dispersed into fine bubbles and mixed with the algal biomass as it is circulated from the bottom of the cell.

The algal biomass can enter the mechanical cell as a feed stream through a feed box. Bubble and algal biomass collisions result from turbulence generated by the rotating impeller. The bubble and algal biomass agglomerates pass out of the collection zone into the separation zone, which is relatively quiescent, where they float to the surface and separate from the liquid phase.

The bubble and algal biomass agglomerates are separated from the liquid phase by gravity and collect as froth concentrated in algal biomass at the top of the cell in the froth zone. Froth concentrated in algal biomass is withdrawn as an algal concentrate stream. The froth normally overflows the cell into a collection launder. Alternatively, the froth may be withdrawn by mechanical means, for example, a froth paddle. The liquid phase is recirculated to the collection zone and eventually exits the cell as a recycle tails stream of brine depleted in algal biomass.

The properly designed rotor and stator mechanism entrains the proper amount of flotation gas, disperses it into fine bubbles, and mixes the flotation gas with liquid to accomplish sufficient contact between the algal biomass and the bubbles. Good mixing and sufficient liquid residence time are necessary in the two phase mixing region to provide high bubble and algae collision efficiency, and good flotation performance.

J_{g} is defined in a mechanical flotation cell as the volumetric gas flow rate divided by the cell cross sectional area parallel to the froth and liquid interface. As the value of J_{g} increases, the gas holdup increases in the liquid phase and decreases in the froth, resulting in potentially faster flotation kinetics but reduced algal biomass concentration in the froth on a flotation gas free basis. The values of J_{g} range from about 0.1 to 5 cm/s for recovery of algal biomass. Values of from about 2 cm/s to 4 cm/s can also be used.

The liquid residence time is defined as the volume of the dispersion in the mechanical cell divided by the volumetric liquid flow rate. Longer residence times enable higher recovery of algal biomass in the froth. The residence time ranges from about 3 to 60, 3 to 30 minutes for continuous operation for the recovery of algal biomass. Residence times greater than 5 minutes can also be employed.

The advantages of a low flotation gas to feed ratio include reduced equipment volume and blower costs in the mechanical cell. The flotation gas to feed ratio ranges from about 1 to 40 for the recovery of algal biomass. Flotation gas to feed ratios of from about 5 to 15 can also be employed.

Impeller tip speed influences the bubble size and the recirculation rate through the collection zone. The bubble size decreases and the recirculation rate through the collection zone increases as the tip speed increases. However, higher tip speeds result in greater mechanical wear and power requirements for the impeller drives. The bubble and algal agglomerates may be broken at high tip speeds. Tip speeds range from about 900 to 2500 feet per minute for the recovery of algal biomass. Tip speeds of from about 1500 to 1800 feet per minute can also be employed.

There are four primary geometrical parameters for mechanical flotation cells. These geometrical parameters are the ratio of rotor submergence to liquid depth, the ratio of tank diameter to impeller diameter, the ratio of liquid depth to tank diameter, and the design of the rotor and stator mechanism. The ratio of rotor submergence to liquid depth ranges from about 0.7 and 0.75 for the recovery of algal biomass. The ratio of tank diameter to impeller diameter ranges from about 1.5 to 5.5. A tank diameter to impeller diameter ratio of about 2 can also be employed. The ratio of liquid depth to tank diameter ranges from about 0.6 to 0.9. A ratio of liquid depth to tank diameter of from about 0.8 to 0.9 can also be employed.

Rotor and stator mechanisms can include those produced by Dorr-Oliver Incorporated of Millford, Conn; Denver Equipment Company which is a division of Svedala of Colorado Springs, Colo.; Wemco Products of Salt Lake City, Utah; and Outotec of Espoo, Finland.

Pneumatic flotation cells differ from mechanically agitated cells in several respects. Bubbles are generated by any nonmechanical means known to the art in a pneumatic cell. Bubbles can be produced by a perforated pipe sparger, an orifice plate, a venturi, or a static mixer. A frother solution usually is mixed with the gas when a static mixer is used.

Some pneumatic cells generate finer bubbles than do mechanical cells. Therefore, the collision frequency is potentially higher, and the residence time required for the flotation is generally shorter in a pneumatic cell.

Pneumatic flotation cells, especially columns, usually have a higher aspect ratio than mechanical cells. The ratio of vessel height to diameter can be greater in the pneumatic cell. It is possible to operate a pneumatic device with a deeper froth bed, allowing for increased drainage time and a drier, more concentrated froth. Wash water can be added to the froth to improve product purity because the vessel height is usually somewhat greater than the vessel diameter. This use of wash water may optionally be used in the instant invention to improve the removal of salt, extraction solvent, and clay from the algal biomass.

Another advantage of a pneumatic flotation cell over a mechanical cell is lighter weight and lower costs of materials and construction. The pneumatic flotation vessel can be constructed of inexpensive lightweight plastics, and weight and cost are reduced by the absence of an impeller and drive. Capital and operating costs for the pneumatic flotation cell may be significantly lower than those for the mechanical cells because no mechanical rotor and stator assembly is required for bubble generation and gas and liquid contacting.

Pneumatic flotation cells can also serve as cleaners operated in either the collection limited regime or in the carrying capacity limited regime. In the collection limited regime, the particle collection rate is limited by the number of collisions between bubbles and the algal biomass. In the carrying capacity limited regime the bubble surfaces are saturated with algal material. Therefore, the particle collection rate is limited by the rate at which bubble surface area is added to the column. It is advantageous to produce a froth whose surface approaches saturation with algal material because it is desirable to minimize the volume of water sent to the downstream process, which may be a drying step.

The flotation gas is dispersed as fine bubbles by means of a bubble generator in a bubble generation zone. The bubble generator may be either internal or external to the froth flotation device. An example of an internal bubble generator is the perforated pipe sparger. An example of an external bubble generator is a static mixer where the gas is mixed with a frother solution.

The bubbles and the algal biomass feed enter the collection zone where bubble and algae collisions occur to form bubble and algal biomass agglomerates. Bubble and algae collisions may be achieved by countercurrent or cocurrent flow of the flotation gas and liquid phases, or by pneumatic mixing. The agglomerates float through the separation zone to the liquid and froth interface and pass into the froth zone where the gas holdup rapidly increases.

The froth may be contacted with wash water to separate entrained hydrophilic particles, for example, salt, or clay from the algal biomass in the froth. The froth leaves the device enriched in algal biomass. The liquid passes through the base of the device as an underflow stream depleted of algal biomass.

Air or an acceptable flotation gas with recycle may be easily used in pneumatic flotation devices. The flotation gas can be recycled by covering the collection launder. Frother may be added either to the liquid phase or to the gas phase to generate small bubbles.

There are several pneumatic flotation devices available that may be used in accordance with the processes and systems disclosed herein to separate salt, extraction solvent and clay from algal biomass. Some of these devices include columns having an aspect ratio greater than one, which provide many of the benefits of the pneumatic devices discussed above.

Suitable pneumatic flotation cells for concentrating algal biomass include, but are not limited to: the air-sparged hydrocyclone, as described in U.S. Pat. No. 4,397,741; the Jameson cell described in U.S. Pat. Nos. 4,938,865, 5,188,726 and 5,332,100; the Canadian column and similar devices with various draft tube designs; the Renewable Algal Energy (RAE) Cell described in WO 2008/156,795 and WO 2008/156,835; and the Microcel™ as defined in U.S. Pat. Nos. 4,981,582 and 5,167,798, the contents of which are incorporated herein by reference in their entirety.

Other adsorptive bubble separation techniques that may be useful in the practice of the dewatering technology include electrolytic flotation and dissolved gas flotation. However, it should be recognized that there are practical limits on these processes and that they are not necessarily equivalent to dispersed gas flotation. In electrolytic flotation, bubbles are generated by passing an electric current through the aqueous medium that is to be separated from the algae. If the aqueous medium is concentrated brine, then a relatively larger current may be needed to generate the bubbles. In dissolved gas flotation, the gas is dissolved in a portion of the feed stream, under pressure in a separate vessel, and the resulting mixture is then introduced into the flotation vessel. The sudden drop in pressure causes the dissolved gas to nucleate and form small bubbles. The solubility of air in brine is somewhat limited and so another, more soluble gas that does not adversely affect algal biomass may be selected, including, for example, helium. These processes may be suitable and useful in certain cases and for specific algal strains.

Suitable gases for use in an adsorptive bubble separation include those that are non-toxic and non-hazardous, for example, air, nitrogen, carbon dioxide, helium, argon and other noble gases, which are generally considered chemically inert, and mixtures thereof. An inert gas that does not contain oxygen or oxidizing agents can also be used to avoid oxidation of the lipids and carotenoids present in the algal cell biomass.

The froth flotation devices used for harvesting described herein can be used in a flotation circuit to maximize recovery and concentration of the valuable components present in the algae. The energy costs for the flotation process are compensated for by the high recovery and concentration factors that may be achieved by using a flotation circuit. Froth flotation devices operate continuously and therefore possess advantages over devices that operate in batch or semi-batch processes. A froth flotation circuit is described in U.S. Pat. No. 5,951,875 for froth flotation columns connected in series of the type that may be used in connection with pneumatic froth flotation. However, it should be understood that the principles represented apply to froth flotation circuits generally, including mechanical and pneumatic froth flotation equipment.

Dissolved air flotation is another suitable adsorptive bubble separation technology for algae and it can be used without or with the addition of flocculating agents. A process using this technique is disclosed in U.S. Pat. No. 4,680,314, the contents of which are incorporated herein by reference in their entirety. This disclosure teaches how Dunaliella or Chlorella can be concentrated with dissolved air flotation after the addition of a flocculating agent, for example, alum or ferric chloride.

Chemical aids may be added to the algal suspension for a variety of purposes as desired or needed prior to dewatering. Chemical aids added to improve separation efficiency may be classified into three categories: agglomerating agents and surface modifying agents. Agglomerating agents, for example, ionic coagulants and polymeric flocculants may be added to generate larger aggregates that are more easily separated. Surface modifying agents, for example, frothers and collectors may be added to render the algae more floatable. Chemical aids could be added in a mixing device after fracking and before the adsorptive bubble separation.

Chemical aids can adversely affect the quality and value of the nutritional ingredients prepared from algal components and of the medium in which the algae are grown. Some chemical additives may not be desirable in processes for producing nutritional ingredients. Disposal problems can arise from the use of chemical aids respecting the algal growth medium and algal residue. For example, undesirable chemical aids are to be avoided in dewatering algal suspensions obtained from the Great Salt Lake when the brine is to be returned to the lake.

Some chemical aids may be undesirable from an economic standpoint. Large quantities of chemicals, for example, alum, are required to coagulate the algal bodies when the algae are obtained from relatively dilute suspensions found in natural lakes and some extensive ponds. A subsequent separation process may be needed to separate the chemical additive from the algal bodies, which increases production cost.

Although not normally necessary, a frother can be used to enhance recovery of the algae in froth flotation processes. A frother may be added to the gas or liquid phase prior to entering the froth flotation device or may be added directly to the algal suspension in the froth flotation device to increase the stability of the froth and to generate small bubbles. Examples of frothers include 2-ethyl hexanol, methyl isobutyl carbinol, which is also known as MIBC, and Dowfroth 250. Dowfroth 250 is a frother that is commercially available from the Dow Chemical Company, which is located in Midland, Mich. When a frother is used, then the frother dosage varies somewhat depending on the manner in which the algal suspension is dewatered. The frother dosage ranges can range from about 5 to 50 ppm.

However, it should be emphasized that no frothers can be used in froth flotation processes described herein. While not wishing to be bound by theory, it is believed that the microalgae contain compounds of sufficient concentration and surfactant power to generate small bubbles in the algal growth medium.

The microalgae in the fracked microalgae stream may be conditioned with collectors and depressors to improve the selectivity of the flotation. For example, it may be desirable to increase the selectivity of the bubbles to adsorb microalgae in preference to halotolerant bacteria or other undesirable competitive algae or predators. Collectors unite with the algal bodies and attach or adsorb them to the bubble surface so that the algal body can be removed with the bubble. On the other hand, depressors unite with the undesirable components present in the suspension to substantially preclude their attachment to a bubble. Use of depressors may be desirable where substantial contaminants would otherwise be recovered with the algae.

Agglomerating agents include synthetic polymers and ionic coagulants. Ionic coagulants can include alum or ferric chloride. Generally, it is desirable to avoid agglomerating agents. Agglomerating agents are not considered necessary in the systems and processes disclosed herein; however, they may be determined to be of benefit in various embodiments, depending on the circumstances, and can be used to advantage. For example, recovery of algal components from deep bed filtration media sometimes can be improved by the use of agglomerating agents.

The algal concentrate stream produced from the algal dewatering unit can be processed by a multiplicity of unit operations, the exact sequence of which will depend on the desired product(s). Suitable unit operations include, but are not limited to pH adjustment, drying, extraction, washing, filtration, dewatering, or combinations thereof. However, for the production of low-value products, for example, biofuels, the algal concentrate can be dried for direct use, or subjected to an extraction step to remove the lipid content prior to the algal biomass being dried.

The algal concentrate stream may include the algal biomass that is recovered in the algal dewatering unit, some residual growth medium, and any compounds that were added during the harvesting or conditioning processes. The algal biomass may include either whole algae or fracked algal cells. If the algal concentrate is to be subjected to the extraction step, the algal cells can be ruptured or fracked prior to the extraction, but it is not essential since some solvents cause cell rupture. If this is not the condition of the algal cells derived from a harvester, the algal cells in the algal concentrate can be disrupted by any process known in the art, for example, that disclosed in U.S. Pat. No. 6,000,551 or in U.S. Pat. No. 8,512,998. Depending on the dewatering efficiency of the harvesting process, the algal concentrate may have a consistency between that of water and of a paste. Preferably, the growth medium will include salts, and it is preferable to remove the unwanted salts prior to drying the algal biomass.

The salts in the algal concentrate stream will be those found in the algal growth medium. These salts can be sodium chloride and the salts that would be encountered via the evaporation of seawater. However, the algal concentrate stream may include salts like sodium carbonate, magnesium chloride, and combinations thereof that are associated with certain saline aquifers and terminal lakes. Herein, salt is defined as any combinations of ions found in ocean water.

After harvesting the optionally conditioned algal concentrate may be passed through a solid-liquid separation unit process for dewatering. The solid-liquid separation unit process may be any process known in the art including a centrifuge, belt press, screw press, filter, cross-flow filter, or combinations thereof. In some embodiments the solid-liquid separation unit process is a centrifuge or a cross-flow microfilter. Centrifuges known in the art may be used in a centrifugal zone to dewater the conditioned algal concentrate, as long as they can readily handle solids.

Suitable centrifuges for the centrifugal zone may be sedimentation centrifuges, filtration centrifuges, hydrocyclones, or combinations thereof. Sedimentation centrifuges may be operated in either batch or continuous mode. Suitable sedimentation centrifuges include, but are not limited to, tubular-bowl centrifuges, multichamber centrifuges, knife-discharge centrifugal clarifiers, disk centrifuges, decanter centrifuges, and screenbowl centrifuges. Specific filtration centrifuges may be operated in either batch or continuous mode. Suitable filtration centrifuges include, but are not limited to pusher centrifuges, conical-screen centrifuges, peeler centrifuges, basket centrifuges, and pendulum centrifuges. In some embodiments, sedimentation centrifuges are more effective at dewatering the conditioned algal concentrate as compared to filtration centrifuges. Disk centrifuges have been found to be effective at dewatering the conditioned algal concentrate in a continuous mode.

Suitable centrifugation equipment can be constructed from acceptable materials of construction in order to handle the ions present in the conditioned algal concentrate. When the pretreated algal concentrate includes sodium chloride, the material of construction of the centrifuge must be appropriate, and materials including, but not limited to stainless steel, Hastelloy, Teflon, plastics, and other materials of construction known in the art to be able to handle the sodium chloride concentration present in the pretreated algal concentrate should be used.

Suitable centrifuges can be those that produce a large enough centrifugal force so that when the residence time of the pretreated algal concentrate in the centrifuge is considered, the pretreated algal concentrate achieves sufficient separation momentum to facilitate the separation of the solids from the liquid phase. Herein, the separation momentum is defined as the force the centrifuge produces multiplied by the residence time of the conditioned algal concentrate in the centrifuge. The force the centrifuge exerts is commonly referred to as a gravitational force and is measured in units of gravitational force. The units of the separation momentum are force·time, commonly g·seconds. Understanding the separation momentum allows similar separation to occur when the centrifuge is scaled. Furthermore, one or more centrifuges may be combined in series or parallel in a manner such that the separation momentum is sufficient to effect the desired separation. The desired and required centrifugal force in g-seconds can be measured in laboratory centrifuges that approximate the g-forces produced in the commercial units. Suitable centrifuges include those manufactured by Alfa-Laval, Westfalia, Andritz, and other similar vendors. Suitable centrifuge types include decanter centrifuges and disc-stack centrifuges that are of either nozzle or shooting bowl design.

Optionally, the algal concentrate being fed to the centrifugal zone can be degassed by any means known in the art, including but not limited to subjecting the algal concentrate to vacuum degassing, ultrasonic degassing, membrane degassing, heating, or combinations thereof.

In exemplary embodiments, the centrifugal zone is operated at a higher pressure than atmospheric so that degassing does not occur in the centrifugal zone. Herein, the centrifuge is operated under sufficient backpressure to prohibit the degassing of the conditioned algal concentrate in the centrifuge. Without wishing to be bound by theory, when the conditioned algal concentrate degasses, gas dissolved in the conditioned algal concentrate is no longer soluble and leaves the solution. When degassing occurs in the centrifuge, gas bubbles may adhere to the algal biomass in the centrifuge, which allows the algal biomass to exit with the lighter aqueous fraction instead of with the heavier semisolid biomass. The backpressure in the centrifuge should be carefully controlled so that it does not cause the bowl of the centrifuge to be forced open, if a disk stack centrifuge with intermittent manual or automatic discharge is being used.

In these embodiments, the treated algal concentrate is fed to the centrifugation zone resulting in the formation of a semisolid biomass that is enriched in algal biomass and aqueous fraction that is depleted in algal biomass, where the semisolid biomass is dewatered treated algal concentrate that is enriched in algal biomass relative to the treated algal concentrate and the aqueous fraction is a liquid depleted in algal biomass relative to the treated algal concentrate. Suitable centrifuges known in the art include, but are not limited to those produced by Westfalia, Bird, and Alfa Laval. Disc centrifuges produced by Westfalia and Alfa Laval are especially preferred.

After passing through the solid-liquid separation unit process the semisolid biomass may be washed in a second solid-liquid separation unit process. Washing occurs by adding fresh water to the semisolid biomass and separating the semisolid biomass from the wash water in a second solid-liquid separation unit process, resulting in washed semisolid biomass. The fresh water is water depleted in salt relative to the semisolid biomass. The washed semisolid biomass is depleted in salt relative to the semisolid biomass and has a lower conductivity than the semisolid biomass.

After harvesting or the centrifugation zone, the washed semisolid biomass may be subjected to an extraction process to recover the lipophilic compounds from the algal biomass. Various streams can optionally be fed to the extraction process including, but not limited to the algal concentrate from the algal dewatering unit, the treated algal concentrate, the semisolid biomass, and combinations thereof.

In exemplary extraction embodiments, the algal concentrate stream is intimately contacted with the extraction solvent that extracts the algal lipids and carotenoids from the algal biomass. The extraction solvent is chosen with the proper polarity so that the desired hydrophobic algal components are preferentially extracted. Thus, the optimal solvent for the extraction process will depend on which lipids or carotenoids are desired to be extracted. Suitable extraction solvents for the extraction include organic solvents, dense gas solvents, aqueous two-phase solvents, ionic liquids, and miscible solvents that phase separate from the algal concentrate stream under certain thermodynamic conditions.

Any solvent system that forms a second immiscible liquid phase with the algal concentrate stream should be useful for extraction of algal oil from the stabilized algal biomass. The solvent system should be one that does not adversely impact the quality or quantity of the algal oil. The solvent system may include synthetic and/or natural flavorants, edible oils, petrochemicals, dense gases, and combinations of these so long as the mixture of the solvent system and the stabilized algal biomass forms two immiscible liquid phases at the desired extraction temperature.

In exemplary embodiments, the solvent system includes petrochemical solvents due to their low viscosity and favorable solute molecular diffusivity. Algal oils can be soluble in petrochemical solvents and concentrated extracts are possible. Suitable petrochemical solvents include those that are disclosed in “Organic Solvents: Physical Properties and Methods of Purification”, edited by J. A. Riddick et al, Volume 2, Fourth Edition, ISBN Number 0-471-08467-0. Petrochemical solvents include: aliphatic hydrocarbons (e.g., pentane, hexane, heptane, octane, nonane, decane, dodecane, cyclohexane, petroleum ether, their isomers, and mixtures thereof), aromatic hydrocarbons (including but not limited to benzene, toluene, xylene), alcohols (including, but not limited to butanol, pentanol, hexanol, octanol, dodecanol, cyclohexanol, benzyl alcohol, their isomers, and combinations thereof), ketones (including, but not limited to methyl isobutyl ketone, hexanone, heptanone, octanone, their isomers, and combinations thereof), esters (including, but not limited to methyl acetate, ethyl acetate, propyl acetate, butyl acetate, pentyl acetate, hexyl acetate, ethyl propionate, ethyl butyrate, ethyl valerate, their isomers, and combinations thereof). Combinations of petrochemical solvents may be used if desired.

In exemplary embodiments, the solvent system includes edible oils that are obtained from plant or animal sources, including fish oils. Edible vegetable oil solvents include, but are not limited to those derived from corn, olive, soybean, flax, safflower, sunflower, palm, jatropha, coconut, other oils known in the art, and combinations thereof. Compared to petrochemical solvents, edible oils can be more viscous, and the solute molecular diffusivity is lower.

In exemplary embodiments, the solvent system includes synthetic and natural flavorants. These solvents can be more desirable than petrochemical solvents and edible oils if the algal oil is to be used for human or animal consumption. Naturally derived flavorants have appeal in nutritional supplements. Flavorants classified by the Flavor and Extract Manufacturers Association, or FEMA, as Generally Recognized As Safe, or GRAS, do not have the drawbacks of petrochemical solvents in association with nutritional supplements. The presence of residual flavorant solvents in nutritional supplements is generally acceptable in comparison with petrochemical solvents, which reduces downstream purification and recovery costs. Flavorants can be selected to have boiling points, viscosities, and molecular diffusivity properties comparable to petrochemical solvents. Examples of flavorants that are suitable for this embodiment include methyl-, ethyl-, propyl-, butyl-, isobutyl-, benzyl-, and octyl-esters with the carboxylic acid component of the ester including acetate, ethanoate, propionate, butyrate, hexanoate, caproate, heptanoate, octanoate, decanoate, cinnamate, and isovalerate. Other examples of flavorants include, but are not limited to, benzaldehyde, other aldehydes, limonene, and other terpenes. Combinations of flavorants may be used, if desired.

In exemplary embodiments, a dense gas is used to extract the algal oil from the algal concentrate stream. Suitable dense gases include, but are not limited to those selected from the group consisting of carbon dioxide, ethane, propane, butane, chlorofluorocarbons, and mixtures thereof. The dense gas extraction may be operated in any manner known in the art, including leaching, batch extraction, and continuous countercurrent extraction as described in U.S. Pat. Nos. 6,106,720 and 5,932,101.

The dense gas may be chosen from a variety of gases, but is preferably carbon dioxide. In addition to being inexpensive and readily available, carbon dioxide is nontoxic, nonflammable, relatively inert, and leaves no algal biomass in the extract. Other suitable dense gases include methane, ethane, propane, butane, isobutane, dimethyl ether, sulfur hexafluoride, ammonia, fluorocarbons, and mixtures thereof.

The dense gas may contain one or more co-solvents to improve extractability of the solute(s). Examples include methanol, ethanol, 1-propanol, 2-propanol, 1-hexanol, 2-methoxy ethanol, acetone, tetrahydrofuran, 1,4-dioxane, acetonitrile, dichloromethane, chloroform, dimethyl sulfoxide, formic acid, carbon disulfide, methylene chloride, amines, chelating agents, phase transfer catalysts and combinations thereof. Other examples of dense gases and co-solvents are listed in U.S. Pat. Nos. 4,345,976 and 5,490,884. The co-solvents may also be added to the fluid feed to enhance recovery of the solute in the extraction solvent.

In exemplary embodiments, the solvent system includes an ionic liquid. Suitable ionic liquids include, but are not limited to solvent systems that are in the liquid phase at the extraction temperature, includes a cation and an anion, and are immiscible with the water-rich algal concentrate phase.

Extraction processes may be operated either in a batch mode or in a continuous mode. Continuous operation can be used for the production of biofuels and can reduce capital and operating costs.

A variety of extraction equipment can be used for continuous extraction processes, the extraction equipment including, but not limited to: mixers and settlers, countercurrent extraction columns, centrifugal extractors, and other classes of extractors known in the art as described by

Pratt et al., Selection, Design, Pilot-Testing, and Scale-Up of Extraction Equipment, Chapter 8, in Science and Practice of Liquid-Liquid Extraction, Volume 1, Clarendon Press, Oxford, 1992, the contents of which are incorporated herein by reference in their entirety. The algal concentrate stream and the extraction solvent may be contacted in countercurrent or concurrent flow.

### Algal Biomass Products

Any of a variety of products can be made from the microorganisms and biomass produced by those microorganisms recovered from the processes described herein, and they include, but are not limited to biofuels, nutraceuticals, cosmeceuticals, wastewater treatment processes, spa products, animal feeds, human food, soil builders, chemical intermediates, specialty lipids, solar salt, and combinations thereof.

Biofuels that may be produced from high temperature processing of the recovered microorganisms and biomass produced by those microorganisms described herein include, but are not limited to biodiesel, green diesel, renewable diesel, methane, alcohols, and dried algal biomass. Algal biodiesel is produced via any transesterification process known in the art, including those which utilize two immiscible liquid phases, and those that utilize a solid acid catalyst. Green diesel may be produced by hydrogenation, cracking, or a combination thereof of the algal oil or any derivative thereof in order to produce hydrocarbons that can be used directly in the existing diesel distribution system. Methane and/or hydrogen may be produced from the recovered microorganisms and biomass produced by those microorganisms by any anaerobic process known in the art. Fermentation of the recovered microorganisms and biomass produced by those microorganisms can be done by any process known in the art used to produce methanol, ethanol, butanol, n-butanol, i-butanol, other alcohols, and combinations thereof. The recovered microorganisms and biomass produced by those microorganisms may be torrified for the production of a soil builder or for use in combination with coal for power or steam generation. The recovered microorganisms and biomass produced by those microorganisms may be dried and then gasified or combusted either by itself or in combination with coal or biomass.

The recovered microorganisms and/or biomass produced by those microorganisms may be extracted to recover the lipids that can be used as an animal feed ingredient, nutraceutical, cosmeceutical, soap or components of a soap or detergent composition, and cosmetic ingredients, including, but not limited to carotenoids, omega fatty acids, and other lipids. For the production of solar salt, the recovered microorganisms and/or biomass produced by those microorganisms may be removed from solar salt works in order to improve the salt quality. The quality of sodium chloride, sodium carbonate, and other salts can be improved by this process. Recovered microorganisms and/or biomass produced by those microorganisms stabilized with a high temperature treatment process may also be used in animal nutrition, especially for shrimp and fish aquaculture diets. Recovered microorganisms and/or biomass produced by those microorganisms may also be treated with the high temperature process in order to stabilize it against degradation during transportation. Alternatively, high temperature processing could be used to stabilize the recovered microorganisms and/or biomass produced by those microorganisms prior to its storage for carbon sequestration purposes. The recovered microorganisms and/or biomass produced by those microorganisms may be used to derive valuable chemical intermediates, for example, fatty acids for the production of polyurethanes.

Suitable animal feeds include, but are not limited to feeds for shrimp, fish, shellfish, brine shrimp, chickens, poultry, cows, ducks, dogs, pigs, sheep, goats, and combinations thereof. The animal feed may require the stabilized recovered microorganisms and/or biomass produced by those microorganisms to be dried, but in some cases, for example for use in shrimp and fish aquaculture diets, complete drying may not be necessary as long as stabilization is sufficient.

Suitable dietary supplements include, but are not limited to alpha carotene, beta-carotene, lutein, zeaxanthin, cryptoxanthin, phytoene, phytofluene, and the various cis- and trans-isomers and the various alpha, beta, gamma, delta isomers of the various carotenoids, and combinations thereof.

Suitable processes of carbon storage include, but are not limited to burying the recovered microorganisms and/or biomass produced by those microorganisms biomass, sinking the recovered microorganisms and/or biomass produced by those microorganisms, torifying the recovered microorganisms and/or biomass produced by those microorganisms and using it as a soil builder, or combinations thereof.

Suitable processes for water and wastewater treatment include, but are not limited to removal of BOD (biological oxygen demand), and or TOC (total organic carbon) from a water stream. This may be useful for municipal wastewater treatment processes, and it may be important for the treatment of brines being used for the production of sodium chloride salt and other salts via evaporation.

Suitable processes to process the recovered microorganisms and/or biomass produced by those microorganisms into useful compounds include, but are not limited to torification, gasification, liquefaction, fermentation, drying, combustion, burial, and combinations thereof. Suitable applications of the torified recovered microorganisms and/or biomass produced by those microorganisms include, but are not limited to a soil builder and a material to be combined with coal, wood, or other combustible material for power generation. Suitable applications of gasified recovered microorganisms and/or biomass produced by those microorganisms include, but are not limited to the production of the entire suite of products that can be produced via syngas chemistry, as described by the Gasification Technologies Council. Suitable products from syngas include, but are not limited to chemicals, fertilizers, power generation, substitute natural gas, hydrogen, and transportation fuels. Suitable chemicals include, but are not limited to hydrogen, carbon monoxide, methanol, dimethyl ether, acetic acid, propionic acid, butyric acid, acetic anhydride, methyl acetate, ethylene, propylene, olefins, and combinations thereof. Suitable fertilizers that can be produced from the syngas include, but are not limited to ammonia, ammonium nitrate, urea, and others known in the art. Suitable substitute natural gas can be generated from the syngas produced by gasifying the recovered microorganisms and/or biomass produced by those microorganisms, and this includes methane. Suitable liquid fuels include gasoline, diesel fuel, jet fuels, and combinations thereof. All of the chemicals that are produced by Eastman Chemicals and by Sasol via their gasification processes may also be produced by the gasification of the recovered microorganisms and/or biomass produced by those microorganisms. Products produced by the utilization of syngas may also be produced by gasification of the recovered microorganisms and/or biomass produced by those microorganisms. Illustrative processes are described in U.S. Pat. No. 6,310,260, the contents of which are incorporated herein by reference in their entirety, include, for example, hydroformylation, hydroacylation (intramolecular and intermolecular), hydrocyanation, hydroamidation, hydroesterification, aminolysis, alcoholysis, hydrocarbonylation, reductive hydroformylation, hydrogenation, olefin oligomerization, hydroxycarbonylation, carbonylation, olefin isomerization, transfer hydrogenation and the like. Preferred processes involve the reaction of organic compounds with carbon monoxide, or with carbon monoxide and a third reactant, e.g., hydrogen, or with hydrogen cyanide, in the presence of a catalytic amount of a metal-organophosphorus ligand complex catalyst.

In exemplary embodiments, the process for maintaining aerobic conditions and/or controlling the algae age in the at least one algal aquaculture zone includes transporting the recovered microorganisms and/or biomass produced by those microorganisms from the harvesting zone to other downstream processing units. The microorganisms and/or biomass produced by those microorganisms processed in the harvesting zone can be sent to a nutrient recovery facility for processing.

When oils are a primary product, some of the recovered microorganisms and/or biomass produced by those microorganisms may be recycled to a nutrient recovery facility where degradation of the microorganisms and/or biomass and the recovery of the valuable growth nutrients from the degraded microorganisms and/or biomass can occur. After degradation of the organic matter (BOD), the nutrient containing solution can be recycled back to the at least one algal aquaculture zone.

Glycerin may also be sent to the nutrient recovery facility after the harvester, and it is termed the polishing pond. In the polishing pond a second microorganism is grown to consume glycerine and other biomass and convert them into carbon dioxide. This CO2 can then be recycled to the algal aquaculture zone as an algal nutrient.

### Systems

The systems for performing the processes described herein can include one or more of the following components: one or more aquaculture zones, the one or more aquaculture zones comprising an aquaculture medium containing microorganism capable of phototaxis; an illumination zone in communication with the one or more aquaculture zones; a separation zone in communication with the one or more aquaculture zones and/or the illumination zone; a harvesting zone in communication with the separation zone; and optionally a lighting system in communication with at least the one or more aquaculture zones, the illumination zone(s) and/or the separation zone.

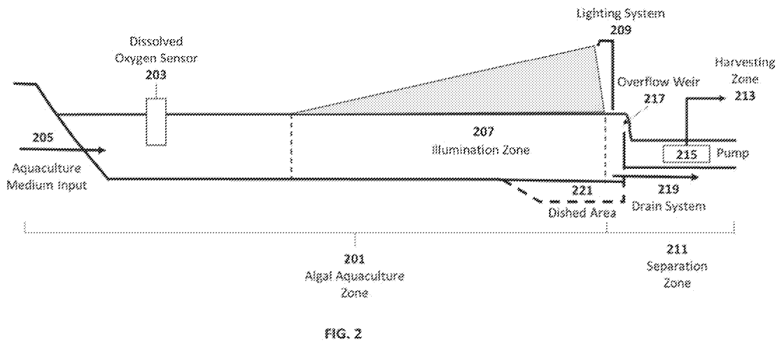

A general overview of one embodiment of the systems for controlling algae age and/or maintaining aerobic conditions in an algal aquaculture zone is disclosed in FIG. 2.

A general overview of one embodiment of the systems for maintaining aerobic conditions in an algal aquaculture zone is disclosed in FIG. 2. The system includes an aquaculture zone 201 containing a dissolved oxygen sensor 203. The system includes an aquaculture medium input pipe, weir or canal 205 configured to control the flow rates of the aquaculture medium into the aquaculture zone 201. The algal aquaculture zone 201 also includes an illumination zone 207 optionally in communication with a lighting system 209.

The lighting system 207 can be used to activate phototaxis of the microorganisms capable of phototaxis in the algal aquaculture zone 201 and/or the illumination zone 207. Activation of phototaxis allows the microorganisms capable of phototaxis to migrate to the upper surface of the aquaculture zone 201 and/or the illumination zone 207. At least part of the upper surface layer is then transported into a separation zone 211 in communication with the aquaculture zone 201 and/or the illumination zone 207, the separation zone 211 also being in communication with a harvesting zone 213. A pump 215 is located within the separation zone and mediates the communication between the separation zone 211 and the harvesting zone 213. The microorganisms capable of phototaxis and/or biomass produced by the microorganisms present in the separation zone 211 is pumped to the harvesting zone 213 for processing.

The system also includes an overflow weir 217 configured to allow the flow of an upper layer of aquaculture medium containing microorganisms capable of phototaxis from the top of the algal aquaculture zone 201 and/or the illumination zone 207 to the separation zone 211.

The system depicted in FIG. 2 can optionally also include drain system 219 configured to allow removal of aquaculture medium from the bottom of the algal aquaculture zone 201 and/or the illumination zone 207. The system can also optionally include a dished area 221 located at the bottom of the algal aquaculture zone 201 and/or the illumination zone 207 near the drain system 219.

The drain system 219 can include an underflow weir and a drainage pipe, tube or canal. The system depicted in FIG. 2 is also configured to remove microorganisms incapable of phototaxis from the bottom of the algal aquaculture zone 201 and/or the illumination zone 207 via the drain system 219. In exemplary embodiments wherein the drainage system 219 contains an underflow weir, the underflow weir can be translocated or moved in a direction that allows the lower layer in the algal aquaculture zone 201 and/or illumination zone 207 to flow into the drainage system 219, wherein the drainage system 219 effectively removes the microorganisms incapable of phototaxis from the aquaculture system.

The microorganisms capable of phototaxis and/or biomass produced by the microorganisms present in the separation zone 211 can be transported to the harvesting zone 213 via the use of pipes, tubes or canals.

In exemplary embodiments, the systems for maintaining aerobic conditions and/or controlling the algae age in an algal aquaculture zone include a filtering unit for separating the microorganisms capable of phototaxis and/or biomass produced by the microorganisms from the bacteria and/or aquaculture medium. The microorganisms capable of phototaxis and/or biomass produced by the microorganisms can be transported to the filtering unit. The filtering unit can be configured to separate the microorganisms capable of phototaxis and/or biomass produced by the microorganisms from the bacteria in the aquaculture medium and can be configured to transport the microorganisms capable of phototaxis and/or biomass produced by the microorganisms to a harvesting zone. In exemplary embodiments, the filtering unit is also configured to transport the bacteria back to the aquaculture zone. The filtering unit can include a filter which is configured to allow bacteria to pass through it but the microorganisms capable of phototaxis and/or biomass produced by the microorganisms. For example, biomass material containing Spirulina algae may be separated from bacteria via this process due to the relatively large size of the Spirulina algae when compared to the bacteria's size.

In an exemplary embodiment of a system wherein a filtering unit is deployed, the filter is selected in accordance to the characteristics of the microalgae being separated from the aquaculture medium. In one embodiment, if Spirulina microalgae are being recovered, a 30 micron filter may be deployed. In another embodiment, if Dunaliella microalgae are being recovered, a cross-flow filter that retains particles greater than 5-10 microns may be deployed.

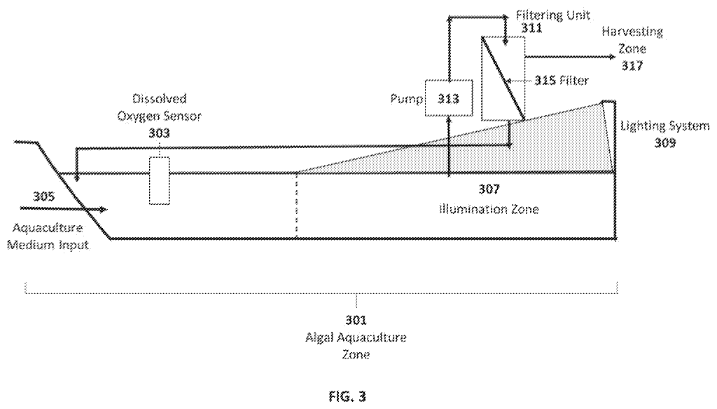

An exemplary embodiment of a system wherein the microorganisms capable of phototaxis and/or biomass produced by the microorganisms are separated from the bacteria via a filtering unit is disclosed in FIG. 3. The aerobic conditions in an algal aquaculture zone 301 are measured by a dissolved oxygen sensor 303. Based on the information collected by the dissolved oxygen sensor 303, the flow rate of the aquaculture medium entering from an aquaculture medium input pipe, weir or canal 305 into the aquaculture zone 301 is controlled. The aquaculture zone 301 also includes an illumination zone 307 optionally in communication with a lighting system 309.

The lighting system 309 can be used to activate phototaxis of the microorganisms capable of phototaxis in the algal aquaculture zone 301 and/or the illumination zone 307. Activation of phototaxis allows the microorganisms capable of phototaxis to migrate to the upper surface of the algal aquaculture zone 301 and/or the illumination zone 307. At least part of the upper surface layer is then transported to a filtering unit 311 via a pump 313. The filtering unit 311 then separates the microorganisms capable of phototaxis and/or biomass produced by the microorganisms from the bacteria present in the transported aquaculture medium via the use of a filter 315. The separated microorganisms capable of phototaxis and/or biomass produced by the microorganisms are then transported to a harvesting zone 317 and the bacteria with at least some of the aquaculture medium are transported back into the algal aquaculture zone 301. The microorganisms capable of phototaxis and/or biomass produced by the microorganisms present in the upper layer of the aquaculture zone 301 and/or illumination zone 307 can be transported to the filtering unit 311 and to the harvesting zone 317 during daylight and/or dark hours. The microorganisms capable of phototaxis and/or biomass produced by the microorganisms present in the upper layer of the algal aquaculture zone 301 and/or illumination zone 309 can be transported to the filtering unit 311 via the use of pipes, tubes or canals. The microorganisms capable of phototaxis and/or biomass produced by the microorganisms present in the filtering unit 311 can be transported to the harvesting zone 317 via the use of pipes, tubes or canals. The aquaculture medium and/or bacteria present in the filtering unit 311 can be transported back to the aquaculture zone 301 via the use of pipes, tubes or canals.

In exemplary embodiments, the systems for maintaining aerobic conditions and/or controlling the algae age in an aquaculture zone include a sedimentation unit for separating the microorganisms capable of phototaxis and/or biomass produced by the microorganisms from the bacteria. The sedimentation unit can be configured to allow the microorganisms capable of phototaxis and/or biomass produced by the microorganisms to settle out of the aquaculture medium. The sedimentation unit can also be configured to transport the microorganisms capable of phototaxis and/or biomass produced by the microorganisms to a harvesting zone. In exemplary embodiments, the sedimentation unit is also configured to transport the bacteria and at least some of the aquaculture medium back to the aquaculture zone.

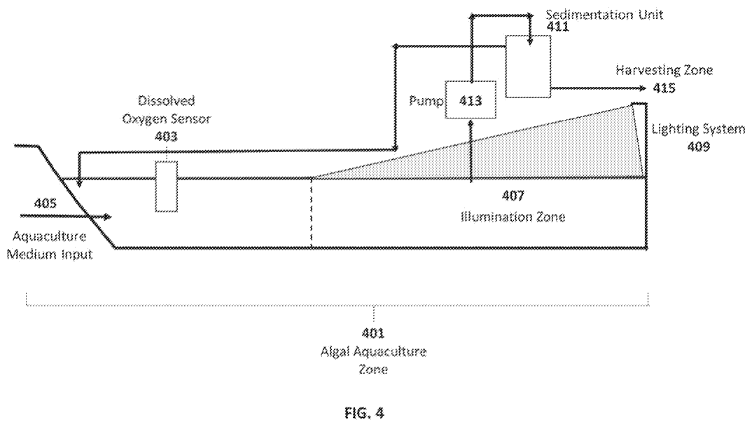

An exemplary embodiment of a system wherein the microorganisms capable of phototaxis and/or biomass produced by the microorganisms is separated from the bacteria via a sedimentation unit is disclosed in FIG. 4. The aerobic conditions in an aquaculture zone 401 are measured by a dissolved oxygen sensor 403. Based on the information collected by the dissolved oxygen sensor 403, the flow rate of the aquaculture medium entering from an aquaculture medium input pipe, weir or canal 405 into the algal aquaculture zone 401 is controlled. The algal aquaculture zone 401 also includes an illumination zone 407 optionally in communication with a lighting system 409.

The lighting system 409 can be used to activate phototaxis of the microorganisms capable of phototaxis in the algal aquaculture zone 401 and/or the illumination zone 407. Activation of phototaxis allows the microorganisms capable of phototaxis to migrate to the upper surface of the algal aquaculture zone 401 and/or the illumination zone 407. At least part of the upper surface is then transported to a sedimentation unit 411 via a pump 413. The sedimentation unit 411 then separates the microorganisms capable of phototaxis and/or biomass produced by the microorganisms from the bacteria present in the transported aquaculture medium by allowing the microorganisms capable of phototaxis and/or biomass produced by the microorganisms to settle out or sediment at the bottom of the sedimentation unit 411. The separated microorganisms capable of phototaxis and/or biomass produced by the microorganisms are then transported to a harvesting zone 415 and the bacteria are transported back into the algal aquaculture zone 401. The microorganisms capable of phototaxis and/or biomass produced by the microorganisms in the upper surface of the algal aquaculture zone 401 and/or the illumination zone 407 can be transported to the sedimentation unit 411 and/or to the harvesting zone 415 during daylight and/or dark hours. The microorganisms capable of phototaxis and/or biomass produced by the microorganisms present in the upper layer of the algal aquaculture zone 401 and/or illumination zone 407 can be transported to the sedimentation unit 411 via the use of pipes, tubes or canals. The microorganisms capable of phototaxis and/or biomass produced by the microorganisms present in the sedimentation unit 411 can be transported to the harvesting zone 415 via the use of pipes, tubes or canals. The aquaculture medium and/or bacteria present in the sedimentation unit 411 can be transported back to the aquaculture zone 401 via the use of pipes, tubes or canals.

In an exemplary embodiment the sedimentation unit is designed to process the desired type of microalgae. In one exemplary embodiment when the microalgae is Dunaliella salina, alum is mixed with the feed to the sedimentation unit so that the algae forms a floc and is separated from the aquaculture medium and sent to the harvesting zone for further processing.

In an exemplary embodiment, the illumination zone is designed so that the phototaxic microorganisms have sufficient residence time in the illumination zone so they can move from the depth of the water column into the upper layer containing microorganism and out of the lower layer before the upper layer is removed by skimming, a weir, or any means known in the art. The desired residence time for the phototaxic microorganisms in the illumination zone is determine by considering the velocity at which the phototaxic microorganism can move and the residence time they have in the illumination zone based on the removal rate of the upper layer containing microorganism. The velocity at which the phototaxic microorganisms can move is experimentally measured at the conditions in the illumination zone. The residence time that the phototaxic microorganisms have in the illumination zone is determined by the system geometry. Thus, the size of the illumination zone is set to provide the phototaxic microorganisms enough time to move from the entire water column in the algal aquaculture zone into the upper layer containing microorganism.

For example, in one exemplary embodiment, a weir is located in the wall of the algal aquaculture pond. When the depth of the illumination zone is 0.5 meters and the phototaxic microorganism can swim at a rate of 0.25 meters per hour, and the flow of the upper layer containing microorganism is 60 cubic meters per hour over the one meter long weir, then the residence time for the algae to exit the bottom half of the depth is one hour. In this case, the width of the illumination zone can be 19 meters (e.g. 9 meters on both sides of the weir plus 1 meter for the weir) and the depth can be 0.5 meters. Thus the width to depth ratio in the illumination zone can be 19/0.5 or 38:1. In exemplary embodiments, the illumination zone or the entirety of the illumination zone has a width to depth ratio from about 100:0.5 to about 1:1 and in some exemplary embodiments a width to depth ratio from about 50:0.5 to 1:0.5.

In exemplary embodiments, the system includes a transition zone configured to allow the transfer of at least part of the upper layer of the illumination zone and/or the aquaculture zone to the separation zone. The transition zone can have a width to depth ratio from about 100:0.5 to about 1:1. In exemplary embodiments, the entirety of the transition zone has a width to depth ratio from about 100:0.5 to about 1:1.

In exemplary embodiments, the system includes a transition zone configured to allow the transfer of at least part of the bottom layer of the illumination zone and/or the aquaculture zone to the separation zone. The transition zone can have a width to depth ratio from about 100:0.5 to about 1:1. In exemplary embodiments, the entirety of the transition zone has a width to depth ratio from about 100:0.5 to about 1:1.

In exemplary embodiments, the system includes at least one dissolved oxygen sensor configured to measure or monitor dissolved oxygen levels in the algal aquaculture zone and/or the illumination with fluorescent-based, galvanic or polarographic means.

In exemplary embodiments, the lighting system includes one or more light sources capable of providing solar radiation and/or artificial light at least to the one or more algal aquaculture zones, the illumination zone, the separation zone and/or the transition zone.

In exemplary embodiments, the one or more algal aquaculture zones are and/or include an open pond, closed pond, raceway pond, tubular reactor, photobioreactor, enclosed raceway, covered pond, open raceway pond, earthen pond, pond in a greenhouse, fermenter, naturally occurring body of water, solar salt pond, or any combinations thereof.

In exemplary embodiments, the one or more algal aquaculture zones contain microorganisms capable of phototaxis. These microorganisms can include species selected from Anabaena, Ankistrodesmus falcatus, Arthrospira (Spirulina) obliquus, Arthrospira (Spirulina) platensis, Botryococcus braunii, Chaetoceros gracilis, Chlamydomonas reinhardtii, Chlorella vulgaris, Chlorella pyrenoidosa, Chlorococcum littorale, Cyclotella cryptica, Dunaliella bardawil, Dunaliella salina, Dunaliella kone, Dunaliella tertiolecta, Dunaliella viridis, Euglena gracilis, Haematococcus pluvialis, Isochrysis galbana, Nannochloris, Nannochloropsis salina, Navicula saprophila, Neochloris oleoabundans, Nitzschia laevis, Nitzschia alba, Nitzschia communis, Nitzschia paleacea, Nitzschia closterium, Nostoc commune, Nostoc flagellaforme, Pleurochrysis carterae, Porphyridium cruentum, Prymnesium, Pseudochoricystis ellipsoidea, Scenedesmus obliquus, Scenedesmus quadricauda, Scenedesmus acutus, Scenedesmus dimorphus, Skeletonema costatum, Spirogyra, Spirulina, Synechoccus, Amphora, Fragilaria, Schizochytrium, Rhodomonas, and genetically-engineered varieties or combinations thereof.

In exemplary embodiments, the system includes one or more weirs optionally located at least partly in the separation zone, the transition zone, the aquaculture zone and/or the illumination zone. The weirs can also be located entirely in the transition zone, the algal aquaculture zone and/or the separation zone. The weirs can be made of materials including, but not limited to wood, cement, plastic, steel, or combinations thereof.

In exemplary embodiments, the transition zone is a canal that possesses an essentially constant width to depth ratio. The width to depth ratio of the canal can range from 100:0.5 to about 1:1.

In exemplary embodiments, the separation zone includes a pump configured to transfer algal growth medium containing microorganisms capable of phototaxis from the separation zone to the harvesting zone. In exemplary embodiments, the pump is located between the separation zone and the harvesting zone.

In exemplary embodiments, the harvesting zone includes, but is not limited to at least one or more of an adsorptive bubble separation unit, a centrifugation unit, a flocculation unit, a sedimentation unit, a filtration unit, and/or any combination thereof.

In exemplary embodiments, the system includes a dished area at least partly located within the one or more aquaculture zones and/or illumination zone, the dished area containing a depth that is greater than the depth of the one or more algal aquaculture zones and/or the illumination zone. In an exemplary embodiment, the dished area can be deeper than the rest of the algal aquaculture zone.

In exemplary embodiments, the one or more aquaculture zones include a paddlewheel configured to provide flow for the microorganisms capable of phototaxis contained within the aquaculture medium.

In exemplary embodiments, the system includes an aquaculture pretreatment unit configured for conditioning an aqueous medium with at least algal nutrients to obtain the aquaculture medium, wherein the aquaculture pretreatment unit is arranged to feed the aquaculture medium to the one or more aquaculture zones, optionally by gravity feed.

In exemplary embodiments, the system includes a pipe, a tube and/or a canal, optionally a covered canal, in communication with the harvesting zone, the illumination zone, the aquaculture zone, the separation zone, the sedimentation unit and/or the filtering unit, wherein the pipe, tube and/or canal is arranged to feed the microorganisms capable of phototaxis and/or biomass produced by those microorganisms to the above zones.

In exemplary embodiments, the system includes a biological polishing system containing a population of microorganisms that metabolize glycerol, optionally in communication with the harvesting zone and/or the one or more aquaculture zones optionally by at least one or more of a pipe, a tube and/or a canal.

In exemplary embodiments, the system includes one or more ponds for crystallizing salt, optionally in communication with the biological polishing system.

In exemplary embodiments, the system includes one or more of the following or any combination thereof: an aquaculture pretreatment unit; a pipe, a tube and/or a canal, optionally a covered canal; a biological polishing system containing a population of microorganisms that metabolize glycerol; one or more ponds for crystallizing salt.

The systems and processes disclosed herein may be applied as an element in a process to cultivate and dewater/harvest algal biomass. The processes to cultivate and dewater/harvest algal biomass may include one or more of the following steps: obtaining water from an appropriate source that may include oceans, rivers, effluent streams, etc.; optionally combining that water with nutrients, inoculum, pest control, and/or recycle streams to obtain an aquaculture medium; culturing algae in an aquaculture pond or a bioreactor; collecting the grown/cultured algae; optionally preparing the algae for dewatering; harvesting or dewatering the algae; obtaining or preparing a stream depleted in algae from the harvesting or dewatering the algae, wherein the stream depleted in algae can be recycled; obtaining the dewatered or harvested algal biomass for downstream processing into valuable components.

The systems and processes disclosed herein may be applied as an element in a system for cultivating and dewatering/harvesting algal biomass, the system for cultivating and dewatering/harvesting algal biomass including one or more of the following: an aquaculture pretreatment unit; a pipe, a tube and/or a canal, optionally a covered canal; a biological polishing system containing a population of microorganisms that metabolize glycerol; one or more ponds for crystallizing salt.

The systems and processes disclosed herein may be applied as an element in a system for culturing algae, wherein the system for culturing algae can include one or more of the following: an aquaculture pretreatment unit configured for conditioning an aqueous medium with at least algal nutrients to obtain the aquaculture medium, wherein the aquaculture pretreatment unit is arranged to feed the aquaculture medium to one or more aquaculture zones described herein optionally by gravity feed; a pipe, a tube and/or a canal, optionally a covered canal, in communication with the harvesting zone, wherein the pipe, tube and/or canal is optionally arranged to feed the microorganisms capable of phototaxis to the harvesting zone; a biological polishing system containing a population of microorganisms that metabolize glycerol, optionally in communication with the harvesting zone and/or the one or more aquaculture zones optionally by at least one or more of a pipe, a tube and/or a canal; one or more ponds for crystallizing salt, optionally in communication with the biological polishing system.

The systems and processes disclosed herein may be applied as an element in a process for culturing algae, wherein the process for culturing algae includes culturing algae in any of the systems disclosed herein.

The systems and processes disclosed herein may be applied as an element in a system configured for culturing and harvesting algae and/or configured for controlling algae age.

The processes disclosed herein can be performed with any of the systems detailed in this disclosure.

The processes and systems disclosed herein can at least be operated in and/or configured for semi-continuous flow. That is, the processes disclosed herein are at least partly operated under continuous flow conditions (e.g., at least one or more of the feeding of the aquaculture medium into the at least one algal aquaculture zone, the exposing the aquaculture medium including microorganisms capable of phototaxis to one or more light sources in the illumination zone of the at least one algal aquaculture zone, the allowing of the microorganisms capable of phototaxis to move toward the one or more light sources in the aquaculture medium to form the upper layer, the separating of at least part of the upper layer from at least part of the lower layer into a separation zone, and the transferring of at least part of the microorganisms capable of phototaxis of the upper layer from the separation zone to a harvesting zone occurs continuously).

In exemplary embodiments, the processes and systems disclosed herein are operated in and/or configured for complete continuous flow. That is, the processes disclosed herein are completely operated under continuous flow conditions.

### Control Aspects

In exemplary embodiments, the process and system disclosed herein can be used to control both the algae age in the algal aquaculture zone and maintaining aerobic conditions in the algal aquaculture zone.

In exemplary embodiments, the process and system disclosed can be used to control the algae age in the algal aquaculture zone. In one exemplary embodiment it is desirable to maintain the algal concentration at a certain level when a continuous flow aquaculture system is deployed. In the case when it is desirable to maintain the algal concentration at a certain level (setpoint), then the amount of algal biomass grown in the algal aquaculture zone on a given day must be removed through the separation zone on that day. By using this invention, the light in the illumination zone can be controlled at night in order to achieve the desired amount of removal based on the measured amount of algal biomass being recovered by the harvester and by monitoring the biomass concentration in the algal aquaculture zone. In a second exemplary embodiment, it is desirable to control the algae age in the algal aquaculture zone. In this case, the concentration of algae flowing into the algal aquaculture zone and the amount of algal growth in the zone is determined and the light in the illumination zone at night is regulated so that the desired amount of algal biomass is removed from the algal aquaculture zone in order to control the algal age in the algal aquaculture zone.

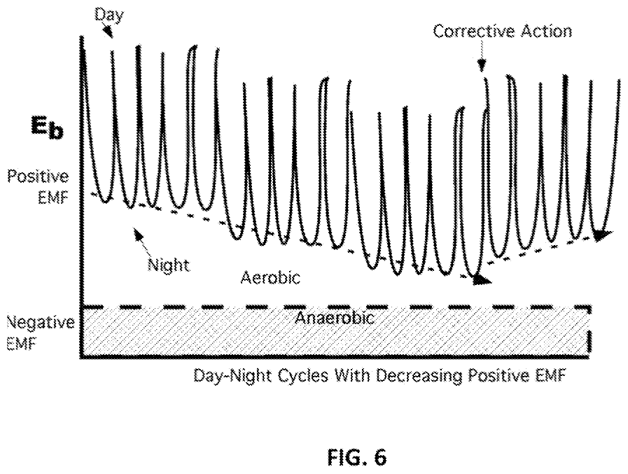

In exemplary embodiments the process and system disclosed can be used to maintain aerobic conditions in the algal aquaculture zone. It is desirable to maintain the dissolved oxygen content above zero at any time during the day or night in order to maintain a healthy algal aquaculture and prevent colony collapse. This occurs when algae and bacteria are competing for an insufficient amount of dissolved oxygen. The presence of additional BOD in the algal aquaculture zone exacerbates the demand on the dissolved oxygen levels. The dissolved oxygen content is continuously measured, and if the levels are dropping as is shown in FIG. 6, then corrective action must be taken to prevent the algal aquaculture zone from becoming anaerobic and risking colony collapse. In one embodiment, the amount of algal biomass entering with the algal growth medium feed can be reduced so that less algal biomass is entering the algal aquaculture zone and thus reducing the demand. If BOD is also entering with the feed stream to the algal aquaculture zone, then reducing the introduction of BOD to the algal aquaculture zone will also reduce the dissolved oxygen demand. In a second embodiment, the light period in the illumination zone can be increased and the flow to the harvester increased in order to remove more algal biomass from the algal aquaculture zone and reduce the dissolved oxygen demand. In a third embodiment, both methods are used in combination.

In all of these embodiments it is desired to return the dissolved oxygen concentration to a pattern as shown in FIG. 6, where the dissolved oxygen level at night drop to a lower level that is still at least 1, 2, 3, 4, 5, 6, 7 mg O2/liter above zero.

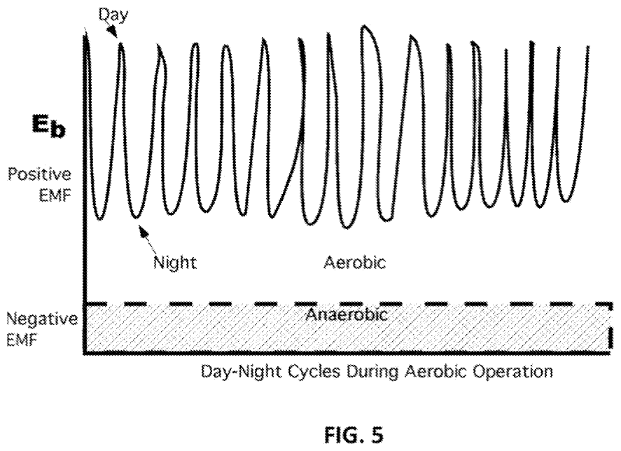

In FIG. 5 and FIG. 6, the y-axis is shown in Eb values and show a positive electromotive force (EMF) which indicates that the algal aquaculture media is aerobic, i.e., the dissolved oxygen content is greater than zero.

### EXAMPLES

### Example 1

Under ideal aerobic operating conditions in an algal culture pond, the oxidation-reduction potential (E_{b}) will remain positive and will not show a drift downward towards anaerobic conditions. This is illustrated in FIG. 5. During the daylight hours, the E_{b} value reaches a maximum, and at night it reaches a minimum. Stable operation is achieved since the electromotive force (EMF) E_{b} never drops into the negative E_{b} anaerobic zone.

### Example 2: Declining EMF with Operational Correction Prior to Anaerobic Conditions

The response time or time constant for a large algal grown facility can be substantial. Therefore, it is necessary to have an early indication that the facility is drifting towards anaerobic conditions. FIG. 6 illustrates a circumstance where there has been a steady decline in the dissolved oxygen concentration in an algal culture pond. Corrective actions can include the purging of the lower layer of the pond and/or a reduction in the quantity of biomass being added to the pond. Either action will leave more oxygen available for algae during periods when they demand oxygen.

### Example 3

An algal aquaculture pond growing Dunaliella algae with earthen construction was 50 hectares in size with the side dimensions of 700 meters in width and 700 meters in length and a mean depth of 0.7 meters. The total volume of the pond was approximately 343,000 cubic meters. There was continuous flow of growth medium that included algae and nutrients into the algal aquaculture pond. There was also continuous flow of growth medium out of the pond through an overflow weir. Thus, the liquid level in the pond was maintained to be constant based on the level of the outlet weir. Both Dunaliella salina and halophylic bacteria were grown in the pond in a mutualistic relationship. The halophylic bacteria and Dunaliella salina were found to be present throughout the water column, except during the afternoon and evening when the Dunaliella salina accumulated in the top layer of the algal aquaculture pond by phototaxis. The Dunaliella salina mean concentration in the algal aquaculture pond was 50,000 cells/ml. This pond was emptied into a receiving pond through an overflow weir that was about 2 meters in length. The semi-circular area in front of the weir was 1.5 meters in depth and this depth region extended about 10 meters in radius from the weir box, and had a volume of about 285 cubic meters. Flow over this weir was about 20 cubic meters per hour, thus the residence time in the quiescent zone was 14.3 hours. The pond width to depth in the quiescent zone was 22 to 1 and at the weir it was 2 to 1. Since the Dunaliella perform phototaxis at a rate of 4.54 cm/hr, they were able to move 65 cm towards the light in the mean residence time in the quiescent zone. The mean concentration of beta-carotene in the algal aquaculture pond throughout the water column was 0.9 ppm. A sample taken of the growth medium flowing over the weir in the morning was about 0.9 ppm beta-carotene concentration with a cell count of about 50,000 cells/ml. However, a sample taken of the growth medium flowing over the weir at 18:00 hours had a beta-carotene concentration of 9 ppm and a cell count of about 510,000 cells/ml. The amount of halophylic bacteria in the samples overflowing the weir were about constant at both times. Thus, when a light was used to prompt algal phototaxis, the algal biomass was preferentially removed from the algal aquaculture pond and separated from the bulk culture medium containing the majority of the halophylic bacterial mass. By encouraging phototaxis, the same algal mass can be removed by skimming the surface layer from the system in a volume that is an order of magnitude less, specifically 10.2x less in this example, than when there was no phototactic skimming. Conversely, if skimming was not used, the flowrate to downstream processing after the weir would need to be increased by an order of magnitude in order to provide the same mean algal biomass residence time in the algal aquaculture pond.

In this particular example the algal residence time in the large growth pond without skimming would be equal to the hydraulic residence time (HRT) or:

$$Algal Biomass Residence time = HRT = V / Q = ( 700 m \times 700 m \times 0.7 m ) / ( 20 {m}^{3} / hr \times 24 hr / day ) = 715 days$$

$$Where V = Pond volume , {m}^{3} and Q = flow out of the pond {m}^{3} / hr$$

However, effective skimming can be controlled to remove more algal biomass within the same time period. In this example, the concentration of algae is 10.3 times the pond concentration. If this concentration is maintained for the 24 hours in which algal biomass is removed, then the algal residence time is decreased by this same factor, or:

$$Algal Biomass Residence time = HRT / Concentration Factor = 715 days / 10.3 = 69 days$$

Therefore, this process was used as an effective means of controlling the algal biomass age in the algal aquaculture pond by purging a more concentrated stream of algae for a measured amount of time that reflects the desired target algal biomass residence time. In addition, this allows downstream processing units to be smaller in size. The algal biomass residence time or algal biomass age is defined as the algal biomass inventory in the aquaculture system in relation to the amount of algal biomass removed from the aquaculture system on a daily basis and can be expressed in units of time.

### Example 4

An algal aquaculture pond growing Dunaliella algae with earthen construction lined with plastic and walls of cinder block was 150 square meters in size with the dimensions of 2.5 meters in width and 60 meters in length around the racetrack pond and a mean depth of 0.3 meters. The total volume of the pond was approximately 45 cubic meters. There was a semi-continuous flow of growth medium that included algae and nutrients into the algal aquaculture pond. There was also continuous flow of growth medium out of the pond through a skimming unit. Both Dunaliella salina and halophylic bacteria were grown in the pond in a symbiotic relationship. The halophylic bacteria and Dunaliella salina were found to be present throughout the water column, except during the afternoon and evening when the Dunaliella salina accumulated in the top layer of the algal aquaculture pond furthest away from the paddlewheel by phototaxis. The Dunaliella salina mean concentration in the algal aquaculture pond was 250,000 cells/ml. A skimming unit was placed at the opposite end of the racetrack-designed pond from the paddlewheel to allow more quiescent flow at the skimming unit. The skimming unit was designed to float on the surface of the pond and remove the top 2-5 millimeters from the top of the pond. From the paddlewheel to the skimming unit there was a length of 50 meters where the aquaculture flow was allowed to stabilize. The paddlewheel was designed to provide minimal agitation for laminar flow so that the algae would have some time to rise to the surface in order to be skimmed. For the daylight hours of 1400 to 1900, the concentration of algae collected in the skimmer averaged 575,000 cells/ml. At other times during the day, the concentration of algae captured in skimming was the mean concentration of the pond. Skimming the pond at this rate removed enough algae from the pond to reduce the mean algal age so that the remaining algal culture needed time to grow before skimming the pond. The amount of halophylic bacteria in the samples overflowing the weir were about constant at both times. Thus, when a light was used to prompt algal phototaxis, the algal biomass was preferentially removed from the algal aquaculture pond and separated from the halophylic bacteria. This process was used to control the mean algal biomass residence time or algae age in the algal aquaculture pond. By encouraging phototaxis, a more concentrated stream of algae by a factor of 2.3, was removed from the racetrack. This effectively reduced the algal biomass residence time by a little over one half as compared to the removal of the same volume but with an algal concentration equivalent to the mean concentration.

In this particular example the algal residence time in the large growth pond without skimming would be equal to the hydraulic residence time (HRT) or:

$$Algal Biomass Residence time = HRT = V / Q = ( 2.5 m \times 60 m \times 0.3 m ) / ( 0.1 {m}^{3} / hr \times 24 hr / day ) = 18.75 days$$

$$Where V = Pond volume , {m}^{3} and Q = flow out of the pond Of 0.1 {m}^{3} / hr$$

However, effective skimming can be controlled to remove more algal biomass within the same time period. In this example, the concentration of algae is 2.3 times the pond concentration for 5 hours/day. If this concentration is maintained for the 5 hours in which algal biomass is removed, then the algal residence time is impacted by this factor, or:

$$Algal Biomass Residence time = [ 18.75 days \times ( 24 - 5 ) + ( 18.75 / 2.3 ) \times 5 ] / 24 = 16.5 days$$

Therefore, this process was used as an effective means of controlling the algal biomass age in the algal aquaculture pond by purging a more concentrated stream of algae for a measured amount of time that reflects the desired target algal biomass residence time.

### Example 5

An algal aquaculture pond growing Dunaliella algae with earthen construction lined with plastic and walls of cinder block was 150 square meters in size with the dimensions of 2.5 meters in width and 60 meters in length around the racetrack pond and a mean depth of 0.3 meters. The total volume of the pond was approximately 45 cubic meters. There was a semi-continuous flow of growth medium that included algae and nutrients into the algal aquaculture pond. There was also continuous flow of growth medium out of the pond through a skimming unit. Both Dunaliella salina and halophylic bacteria were grown in the pond in a mutualistic relationship. The halophylic bacteria and Dunaliella salina were found to be present throughout the water column, except during the afternoon and evening when the Dunaliella salina accumulated in the top layer of the algal aquaculture pond furthest away from the paddlewheel by phototaxis. The Dunaliella salina mean concentration in the algal aquaculture pond was 200,000 cells/ml (cpm). A skimming unit was placed at the opposite end of the racetrack-designed pond from the paddlewheel to allow more quiescent flow at the skimming unit. The skimming unit was designed to float on the surface of the pond and remove the top 1-3 millimeters from the top of the pond. From the paddlewheel to the skimming unit there was a length of 50 meters where the aquaculture flow was allowed to stabilize. The paddlewheel was designed to provide minimal agitation for laminar flow so that the algae would have some time to rise to the surface in order to be skimmed. For the daylight hours of 1300 to 1800, the concentration of algae collected in the skimmer averaged 1,000,000 cells/ml (cpm). At other times during the day, the concentration of algae captured in skimming was the mean concentration of the pond. Skimming the pond at this rate removed enough algae from the pond to reduce the mean algal age so that the remaining algal culture needed time to grow before any subsequent skimming of the pond. The amount of halophylic bacteria in the samples overflowing the weir were about constant at both times. Thus, when a light was used to prompt algal phototaxis, the algal biomass was preferentially removed from the algal aquaculture pond and separated from the halophylic bacteria. As in Examples 1 and 2, this process was used to control the algal biomass residence time or algae age in the algal aquaculture pond by increasing the algal biomass concentration by a factor of 1,000,000 cpm/200,000 cpm=5.

In this particular example the algal residence time in the large growth pond without skimming would be equal to the hydraulic residence time (HRT) or:

$$Algal Biomass Residence time = HRT = V / Q = ( 2.5 m \times 60 m \times 0.3 m ) / ( 0.1 {m}^{3} / hr \times 24 hr / day ) = 18.75 days$$

$$Where V = Pond volume , {m}^{3} and Q = flow out of the pond Of 0.1 {m}^{3} / hr$$

However, effective skimming can be controlled to remove more algal biomass within the same time period. In this example, the concentration of algae is 2.3 times the pond concentration for 5 hours/day. If this concentration is maintained for the 5 hours in which algal biomass is removed, then the algal residence time is impacted by this factor, or:

$$Algal Biomass Residence time = [ 18.75 days \times ( 24 - 5 ) + ( 18.75 / 2.3 ) \times 5 ] / 24 = 16.5 days$$

Therefore, this process was used as an effective means of controlling the algal biomass age in the algal aquaculture pond by purging a more concentrated stream of algae for a measured amount of time that reflects the desired target algal biomass residence time.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

1. A process to culture algae by controlling algae age and/or maintaining aerobic conditions in an algal growth system, the process comprising:
feeding an aquaculture medium including microorganisms capable of phototaxis into at least one algal aquaculture zone, wherein the microorganisms include algae;
exposing the aquaculture medium to one or more light sources in an illumination zone of the at least one algal aquaculture zone, wherein the entirety of the illumination zone has a width to depth ratio from about 100:0.5 to about 1:1;
allowing the microorganisms capable of phototaxis to move toward the one or more light sources in the aquaculture medium, thereby forming an upper layer containing the microorganisms capable of phototaxis and a lower layer in the illumination zone, wherein the upper layer is 1 to 100 mm deep;
separating at least part of the upper layer from at least the lower layer in a separation zone; and
transferring at least part of the microorganisms capable of phototaxis of the upper layer from the separation zone to a harvesting zone.

2. The process of claim 1, wherein the algal growth system comprises a transition zone allowing a transfer of at least part of the upper layer of the illumination zone to the separation zone and optionally the entirety of the transition zone has a width to depth ratio from about 100:0.5 to about 1:1.

3. The process of claim 2, wherein the transition zone is a canal that optionally possesses the width to depth ratio essentially constant.

4. The process of claim 1, wherein the separating comprises skimming at least part of the upper layer into the separation zone, optionally over one or more weirs.

5. The process of claim 4, wherein the one or more weirs are made of wood, plastic, steel, cement or combinations thereof.

6. The process of claim 1, wherein the process comprises measuring and/or monitoring dissolved oxygen levels in the at least one algal aquaculture zone and/or the illumination zone.

7. The process of claim 6, wherein the measuring and/or monitoring of dissolved oxygen levels is performed by at least one dissolved oxygen sensor configured to measure and/or monitor dissolved oxygen levels optionally with fluorescent-based, galvanic or polarographic means.

8. The process of claim 7, wherein the at least one dissolved oxygen sensor measures galvanic current and/or tracks millivoltage levels in the at least one aquaculture zone and/or the illumination zone.

9. The process of claim 1, the process comprising maintaining oxidation-reduction potentials in the at least one algal aquaculture zone and/or the illumination zone at Eh values above 0 V.

10. The process of claim 1, the process comprising maintaining oxidation-reduction potentials in the at least one aquaculture zone and/or the illumination zone before separating at least part of the upper layer from at least the lower layer in the separation zone.

11. The process of claim 1, the process comprising determining the dissolved oxygen level in the at least one aquaculture zone and/or the illumination zone, and optionally maintaining it above 0 mg/l.

12. The process of claim 1, wherein the exposing to the one or more light sources occurs from about 9 hours to about 24 hours a day.

13. The process of claim 1, wherein the one or more light sources include solar radiation and/or an artificial light source.

14. The process of claim 1, wherein the at least one algal aquaculture zone comprises or consists of a pond, optionally an open pond.

15. The process of claim 1, wherein the microorganisms capable of phototaxis include one or more selected from the group comprising or consisting of Anabaena, Ankistrodesmus falcatus, Arthrospira (Spirulina) obliquus, Arthrospira (Spirulina) platensis, Botryococcus braunii, Chaetoceros gracilis, Chlamydomonas reinhardtii, Chlorella vulgaris, Chlorella pyrenoidosa, Chlorococcum littorale, Cyclotella cryptica, Dunaliella bardawil, Dunaliella salina, Dunaliella kone, Dunaliella tertiolecta, Dunaliella viridis, Euglena gracilis, Haematococcus pluvialis, Isochrysis galbana, Nannochloris, Nannochloropsis salina, Navicula saprophila, Neochloris oleoabundans, Nitzschia laevis, Nitzschia alba, Nitzschia communis, Nitzschia paleacea, Nitzschia closterium, Nostoc commune, Nostoc flagellaforme, Pleurochrysis carterae, Porphyridium cruentum, Prymnesium, Pseudochoricystis ellipsoidea, Scenedesmus obliquus, Scenedesmus quadricauda, Scenedesmus acutus, Scenedesmus dimorphus, Skeletonema costatum, Spirogyra, Spirulina, Synechoccus, Amphora, Fragilaria, Schizochytrium, Rhodomonas, and genetically-engineered varieties or combinations thereof.

16. The process of claim 1, wherein the aquaculture medium and/or the at least one algal aquaculture zone contains microorganisms

17. The process of claim 1, wherein the algal growth system comprises a pump configured to transfer at least part of the microorganisms capable of phototaxis of the upper layer from the separation zone to the harvesting zone.

18. The process of claim 1, wherein the harvesting zone comprises at least one or more of an adsorptive bubble separation unit, a centrifugation unit, a flocculation unit, a sedimentation unit, a filtration unit, and/or any combination thereof.

19. The process of claim 1, the process comprising harvesting at least part of the microorganisms capable of phototaxis of the upper layer in the harvesting zone.

20. The process of claim 19, wherein the harvesting comprises at least one or more of rupturing the microorganisms capable of phototaxis, performing adsorptive bubble separation, centrifuging the microorganisms or ruptured microorganisms, flocculating the microorganisms or ruptured microorganisms, sedimenting the microorganisms or ruptured microorganisms, filtering the microorganisms or ruptured microorganisms, and/or any combination thereof.

21. An algal growth system for culturing algae by controlling algae age and/or maintaining aerobic conditions in the system, the system comprising:
one or more algal aquaculture zones, the one or more algal aquaculture zones including an aquaculture medium containing microorganisms capable of phototaxis;
an illumination zone in communication with the one or more algal aquaculture zones, wherein the entirety of the illumination zone has a width to depth ratio from about 100:0.5 to about 1:1;
a separation zone in communication with the one or more algal aquaculture zones;
a harvesting zone in communication with the separation zone; and
optionally a lighting system in communication with at least the one or more algal aquaculture zones, the illumination zone and/or the separation zone.

22. The system of claim 21, wherein the algal growth system comprises a transition zone allowing a transfer of at least part of the upper layer of the illumination zone to the separation zone and optionally the entirety of the transition zone has a width to depth ratio from about 100:0.5 to about 1:1.

23. The system of claim 21, wherein the system comprises at least one dissolved oxygen sensor configured to measure and/or monitor dissolved oxygen levels with fluorescent-based, galvanic or polarographic means.

24. The system of claim 23, wherein the at least one dissolved oxygen sensor measures galvanic current and/or tracks millivoltage levels in the one or more algal aquaculture zones.

25. The system of claim 23, wherein the at least one dissolved oxygen sensor is configured to measure oxidation-reduction potentials in the one or more algal aquaculture zones.

26. The system of claim 21, wherein the lighting system includes one or more light sources capable of providing solar radiation and/or artificial light to at least the one or more algal aquaculture zones and/or the transition zone.

27. The system of claim 21, wherein the one or more algal aquaculture zones comprises or consists of a pond, optionally an open pond.

28. The system of claim 21, wherein the microorganisms capable of phototaxis include one or more selected from the group comprising or consisting of Anabaena, Ankistrodesmus falcatus, Arthrospira (Spirulina) obliquus, Arthrospira (Spirulina) platensis, Botryococcus braunii, Chaetoceros gracilis, Chlamydomonas reinhardtii, Chlorella vulgaris, Chlorella pyrenoidosa, Chlorococcum littorale, Cyclotella cryptica, Dunaliella bardawil, Dunaliella salina, Dunaliella kone, Dunaliella tertiolecta, Dunaliella viridis, Euglena gracilis, Haematococcus pluvialis, Isochrysis galbana, Nannochloris, Nannochloropsis salina, Navicula saprophila, Neochloris oleoabundans, Nitzschia laevis, Nitzschia alba, Nitzschia communis, Nitzschia paleacea, Nitzschia closterium, Nostoc commune, Nostoc flagellaforme, Pleurochrysis carterae, Porphyridium cruentum, Prymnesium, Pseudochoricystis ellipsoidea, Scenedesmus obliquus, Scenedesmus quadricauda, Scenedesmus acutus, Scenedesmus dimorphus, Skeletonema costatum, Spirogyra, Spirulina, Synechoccus, Amphora, Fragilaria, Schizochytrium, Rhodomonas, and genetically-engineered varieties or combinations thereof.

29. The system of claim 21, wherein the aquaculture medium and/or the one or more algal aquaculture zones include microorganisms capable of growing at a salinity from about 5 wt % to about 27 wt %.

30. The system of claim 21, wherein the system comprises one or more weirs optionally located at least partly in the separation zone and/or in the transition zone.

31. The system of claim 30, wherein the one or more weirs are made of wood, cement, plastic, steel, or combinations thereof.

32. The system of claim 22, wherein the transition zone is a canal that possesses the width to depth ratio essentially constant.

33. The system of claim 21, wherein the separation zone comprises a pump configured to transfer microorganisms capable of phototaxis from the separation zone to the harvesting zone.

34. The system of claim 21, wherein the harvesting zone comprises at least one or more of an adsorptive bubble separation unit, a centrifugation unit, a flocculation unit, a sedimentation unit, a filtration unit, and/or any combination thereof.

35. The system of claim 21, wherein the system comprises a dished area at least partly located within the one or more algal aquaculture zones, the dished area containing a depth that is greater than the depth of the one or more algal aquaculture zones.

36. The system of claim 21, wherein the one or more algal aquaculture zones comprise a paddlewheel configured to provide flow for the microorganisms capable of phototaxis.

37. The process or system of claim 1, wherein the algal growth system comprises:
an aquaculture pretreatment unit configured for conditioning an aqueous medium with at least algal nutrients to obtain the aquaculture medium, wherein the aquaculture pretreatment unit is arranged to feed the aquaculture medium to the one or more algal aquaculture zones optionally by gravity feed;
a pipe, a tube and/or a canal, optionally a covered canal, in communication with the harvesting zone, wherein the pipe, tube and/or canal is arranged to feed the microorganisms capable of phototaxis to the harvesting zone;
a biological polishing system containing a population of microorganisms that metabolize glycerol, optionally in communication with the harvesting zone and/or the one or more algal aquaculture zones optionally by at least one or more of a pipe, a tube and/or a canal; and/or
one or more ponds for crystallizing salt, optionally in communication with the biological polishing system.

38. The system of claim 21, wherein the system performs the process of any of claim 1-20 or 37.

39. A process for culturing and harvesting algae, the process comprising using the system of claim 21.

40. Use of the system of claim 21 for culturing and harvesting algae and/or for controlling algae age.
